(19) United States
(12) Patent Application Publication
(10) Pub. No.: US 2026/0345720 A1
(43) Pub. Date: Nov. 5, 2026

(54) COMPOSITIONS, DRUG PRODUCTS, METHODS, AND RELATED USES FOR ADDRESSING DRY AGE-RELATED MACULAR DEGENERATION

(71) Applicant: Stealth BioTherapeutics Inc., Needham, MA (US)

(72) Inventors: Anthony Abbruscato, Needham, MA (US); Michael DiMatteo, Needham, MA (US); James Wakefield, Needham, MA (US); John Ciallella, Needham, MA (US); Naidong Ye, Ponte Vedra, FL (US)

(73) Assignee: Stealth BioTherapeutics Inc., Needham, MA (US)

(21) Appl. No.: 19/659,729

(22) Filed: Apr. 27, 2026

Related U.S. Application Data
(60) Provisional application No. 63/794,922, filed on Apr. 25, 2025.

(51) Int. Cl. A61K 38/06; A61K 9/00; A61K 47/02

(52) U.S. Cl. CPC A61K 38/06; A61K 9/0048; A61K 47/02

(57) ABSTRACT

The present disclosure relates to compositions, drug products, methods, and related uses for dosing bevemipretide, or a pharmaceutically acceptable salt thereof, in subjects in need thereof, such as for addressing an ophthalmic medical indication. The present disclosure further relates to compositions, drug products, methods, and related uses for treating, preventing, inhibiting, ameliorating, or delaying the onset of dry age-related macular degeneration, wherein all of the foregoing pertain to dosing bevemipretide, or a pharmaceutically acceptable salt thereof, in subjects in need thereof.

### CROSS-REFERENCE TO RELATED APPLICATIONS

This application claims the benefit of and priority to U.S. Provisional Patent Application No. 63/794,922, filed Apr. 25, 2025, the entire contents of which are incorporated herein by reference for any and all purposes.

### TECHNICAL FIELD

The present technology relates generally to compositions and drug products (including combination drug products) that are, inter alia, suitable for ophthalmic topical administration/dosing of bevemipretide, or a pharmaceutically acceptable salt thereof, to a subject and related methods and uses. For example, the present technology relates to compositions, drug products (including combination drug products), related uses, and methods suitable for treating, preventing, inhibiting, ameliorating, or delaying the onset of dry age-related macular degeneration wherein bevemipretide, or a pharmaceutically acceptable salt thereof, is administered as an ophthalmic topical solution to a subject in need thereof, such as by administration as an eye drop.

### INTRODUCTION

The following description is provided to assist the understanding of the reader. None of the information provided or references cited is admitted as being prior art to the present technology.

Mitochondrial dysfunction is an integral component of disease etiology in dry age-related macular degeneration (AMD). See, e.g., Kaarniranta, et al., Prog Retin Eye Res. 2020 November;79:100858. Elamipretide, a mitochondria-targeted therapeutic, is being investigated in two global Phase 3 clinical trials for the treatment of dry AMD. See Ehlers, et al., Ophthalmol Sci. 2024 Oct. 9;5 (1): 100628; and Mettu, et al.,. Opththalmol Sci. 2022; 2:100086. Bevemipretide (a.k.a., SBT-272 or (R)-2-amino-N-((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide), is a next-generation mitochondria-targeting therapeutic that restores mitochondrial structure and function. Non-human animal studies have demonstrated penetration of bevemipretide to critical retinal tissues through administration of a topical eye drop formulation. See Ciallella, et al., Poster session presented at: Keystone Symposium: Dry AMD: Pathophysiology and Emerging Technologies. Sep. 17-20, 2024; Pacific Grove, CA. It has been reported that bevemipretide can be used to address ophthalmic indications, including diseases or conditions affecting the retina or other ocular tissues (see, e.g., International Patent Publication No. WO 2023/069549, which describes the use of bevemipretide in the treatment of ophthalmic disease). Methods, compositions, combination drug products and related uses that treat, prevent, inhibit, ameliorate, or delay the onset of dry age-related macular degeneration represent important advances in patient care that are much needed.

### SUMMARY

In some embodiments, this technology is directed to an ophthalmic topical composition comprising about 1% to about 3% bevemipretide or a pharmaceutically acceptable salt thereof, where the stated “percent bevemipretide” is based on the free-base weight of the bevemipretide to volume (w/v) of the composition, about 1.4 mg/mL to about 5.4 mg/mL sodium chloride (NaCl), about 4.1 mg/mL to about 4.2 mg/mL sodium phosphate monobasic monohydrate (NaH₂PO₄·H₂O), and water (generally-water for injection); wherein a pH of the ophthalmic topical composition is in the range of 5.7 to 5.9, inclusive. In preparation of this composition, generally reagents (e.g., bevemipretide, NaCl, and NaH₂PO₄·H₂O) are dissolved in less than the final volume/mass of water, mixed until everything has been dissolved and evenly distributed throughout. If needed the pH can be adjusted with, for example, hydrochloric acid or sodium hydroxide to achieve the desired pH and then additional water is added until the final volume or mass is achieved. The composition can be prepared volumetrically or gravimetrically. The composition can be preservative-free, sterile or preservative-free and sterile. Thus, in some embodiments, this technology is further directed to a preservative-free ophthalmic topical composition, the composition comprising an aqueous solution prepared by the dissolution in water (generally water for injection); about 1% to about 3% bevemipretide or a pharmaceutically acceptable salt thereof, where the stated “percent bevemipretide” is based on the free-base weight of the bevemipretide to volume (w/v) of the composition, about 1.4 mg/mL to about 5.4 mg/mL sodium chloride (NaCl), about 4.1 mg/mL to about 4.2 mg/mL sodium phosphate monobasic monohydrate (NaH₂PO₄·H₂O), and sufficient sodium hydroxide and/or hydrochloric acid to maintain a desired pH of the composition; wherein the desired pH of the ophthalmic topical composition is in the range of 5.7 to 5.9, inclusive.

In some embodiments, the ophthalmic topical composition comprises about 1% bevemipretide, about 5.3 mg/mL NaCl, and about 4.16 mg/mL NaH₂PO₄·H₂O, wherein the pH is about 5.8. In some embodiments, the ophthalmic topical composition comprises about 2% bevemipretide, about 3.4 mg/mL NaCl, and about 4.16 mg/mL NaH₂PO₄·H₂O, wherein the pH is about 5.8. In some embodiments, the ophthalmic topical composition comprises about 3% bevemipretide, about 1.5 mg/mL NaCl, and about 4.16 mg/mL NaH₂PO₄·H₂O, wherein the pH is about 5.8.

In some embodiments, the ophthalmic topical composition is sterile.

In some embodiments of the foregoing ophthalmic topical composition, essentially no other reagents or ingredients are required to produce a suitable pharmaceutical product. Accordingly, in some embodiments, this technology is directed to ophthalmic topical composition consisting essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water (generally water for injection), wherein a pH of the ophthalmic topical composition is in the range of 5.7 to 5.9, inclusive. The composition can be prepared volumetrically or gravimetrically.

In some embodiments, the ophthalmic topical composition consists essentially of about 1% bevemipretide, about 5.3 mg/mL NaCl, and about 4.16 mg/mL NaH₂PO₄·H₂O, wherein the pH is about 5.8. In some embodiments, the ophthalmic topical composition consists essentially of about 2% bevemipretide, about 3.4 mg/mL NaCl, and about 4.16 mg/mL NaH₂PO₄·H₂O, wherein the pH is about 5.8. In some embodiments, the ophthalmic topical composition consists essentially of about 3% bevemipretide, about 1.5 mg/mL NaCl, and about 4.16 mg/mL NaH₂PO₄·H₂O, wherein the pH is about 5.8.

In some embodiments, the ophthalmic topical composition is sterile. In some embodiments, the ophthalmic topical composition is preservative-free. In some embodiments, the ophthalmic topical composition is sterile and preservative-free.

The ophthalmic topical composition previously described can be packaged into a device for administration. Such device can be for single administration (e.g., single-use) device or multi-administration (e.g., multi-day/multi-dose). Because such packaged solution is in a form suitable for administration to, or by, a subject, it is sometimes referred to here as a “drug product”. Such device can be a container adapted for such single-use or multi-dose administration. One such container can be a blow-fill-seal (BFS) vial; which vial can be manufactured, filled and sealed in a continuous process in a suitably designed machine. Alternatively, a multi-dose container can be used. In some embodiments, the multi-dose container is adapted to maintain the sterility of the ophthalmic topical solution through repeated dosing over days, weeks, months or even years.

Thus, in some embodiments, this technology is directed to a packaged ophthalmic topical composition comprising a blow-fill-seal (BFS) vial, wherein the blow-fill-seal vial comprises a composition comprising about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive. In some embodiments, the packaged ophthalmic topical composition comprises about 1% bevemipretide, about 5.3 mg/mL NaCl, and about 4.16 mg/mL NaH₂PO₄·H₂O, wherein the pH is about 5.8. In some embodiments, the packaged ophthalmic topical composition comprises about 2% bevemipretide, about 3.4 mg/mL NaCl, and about 4.16 mg/mL NaH₂PO₄·H₂O, wherein the pH is about 5.8. In some embodiments, the packaged ophthalmic topical composition comprises about 3% bevemipretide, 1.5 mg/mL NaCl, and about 4.16 mg/mL NaH₂PO₄·H₂O, wherein the pH is about 5.8.

The characteristics/features of the BFS vial can be controlled by design of a mold used to fashion the BFS container from an extruded resin. Thus, in some embodiments, the volume of the blow-fill-seal vial of the packaged ophthalmic topical composition is in the range of 0.5 mL to 1.0 mL, inclusive. In some embodiments, the target volume of the blow-fill-seal vial of the packaged ophthalmic topical composition is about 0.8 mL.

The volume of liquid (e.g., ophthalmic topical composition) sited in (e.g., injected into) the BFS vial during the manufacturing process can be controlled by the equipment design and adjustment of various parameters of machine operation, all of which is well-understood and easily controlled. Thus, in some embodiments, the volume of the composition in the blow-fill-seal vial of the packaged ophthalmic topical composition is in the range of 0.24 mL to 0.40 mL, inclusive. In some embodiments, the target volume of the composition in the blow-fill-seal vial of the packaged ophthalmic topical composition is about 0.32 mL.

The mold used to fashion the container in the BFS equipment can be designed to create and fill a multiple vial article (such as a multi-vial card), thereby creating and filling multiple vials simultaneously. Therefore, in some embodiments, the blow-fill-seal vial of the packaged composition is linked physically to one more additional blow-fill-seal vials also containing the ophthalmic topical composition. For example, the blow-fill-seal vial can be linked physically to 4 additional blow-fill-seal vials also containing the ophthalmic topical composition to create a 5-unit article of the packaged ophthalmic topical composition; an illustration of one such embodiment is presented in FIG. 5. In some embodiments, the blow-fill-seal vial can be linked physically to 6 additional blow-fill-seal vials also containing the ophthalmic topical composition to thereby create a 7-unit article of the packaged ophthalmic topical composition that would be suitable as a one week (7-day) supply of drug product if dosing/administration occur once per day. In some embodiments, the blow-fill-seal vial can be linked physically to 13 additional blow-fill-seal vials also containing the ophthalmic topical composition to thereby create a 14-unit article of the packaged ophthalmic topical composition that would be suitable as a two week (14 day) supply of drug product if dosing/administration occur once per day or a one week (7 day supply) if dosing/administration occur twice per day. In some embodiments, the blow-fill-seal vial can be linked physically to 27 additional blow-fill-seal vials also containing the ophthalmic topical composition to thereby create a 28-unit article of the packaged ophthalmic topical composition that would be suitable as a 28-day supply of drug product if dosing/administration occur once per day or a two week (14 day supply) if dosing/administration occur twice per day. In some embodiments, the blow-fill-seal vial can be linked physically to 29 or 30 additional blow-fill-seal vials also containing the ophthalmic topical composition to create a 30 or 31-unit article of the packaged ophthalmic topical composition that would be suitable as a one month supply of drug product if dosing/administration occur once per day. In some embodiments, the blow-fill-seal vial is linked physically to 1 to 30 additional blow-fill-seal vials also containing the ophthalmic topical composition to create a 2 to 31 unit article.

In some embodiments, the blow-fill-seal vial of the packaged ophthalmic topical composition comprises low density polyethylene (LDPE). In some embodiments, the blow-fill-seal vial of the packaged ophthalmic topical composition comprises a twist off cap. In some embodiments of the packaged ophthalmic topical composition, the composition is sterile. In some embodiments of the packaged ophthalmic topical composition, the composition is preservative-free. In some embodiments of the packaged ophthalmic topical composition, the composition is sterile and preservative-free. In some embodiments, the packaged ophthalmic topical composition is adapted for single use.

As mentioned above, the ophthalmic topical composition can be packed in a multi-dose container. Thus, this technology relates to a packaged ophthalmic topical composition comprising a multi-dose container, wherein the multi-dose container comprises a composition comprising about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive. In some embodiments, the packaged ophthalmic topical composition comprises about 1% bevemipretide, about 5.3 mg/mL NaCl, and about 4.16 mg/mL NaH₂PO₄·H₂O, wherein the pH is about 5.8. In some embodiments, the packaged ophthalmic topical composition comprises about 2% bevemipretide, about 3.4 mg/mL NaCl, and about 4.16 mg/mL NaH₂PO₄·H₂O, wherein the pH is about 5.8. In some embodiments, the packaged ophthalmic topical composition comprises about 3% bevemipretide, 1.5 mg/mL NaCl, and about 4.16 mg/mL NaH₂PO₄. H₂O, wherein the pH is about 5.8.

In some embodiments of the multi-dose packaged ophthalmic topical composition, the composition is sterile. In some embodiments of the multi-dose packaged ophthalmic topical composition, the composition is preservative-free. In some embodiments of the multi-dose packaged ophthalmic topical composition, the composition is sterile and preservative-free.

In some embodiments of the packaged ophthalmic topical composition, the multi-dose container is adapted to maintain sterility of the composition for a week or more, a month or more, three months or more, 6 months or more or a year or more. In some embodiments of the packaged ophthalmic topical composition, the multi-dose container is adapted to comprise between 2.5 mL and 30 mL, inclusive, of ophthalmic topical composition. In some embodiments of the packaged ophthalmic topical composition, the multi-dose container is adapted to deliver a consistent dose of between 25 μL and 35 μL, inclusive, as a drop of ophthalmic topical solution each time it is used. In some embodiments of the packaged ophthalmic topical composition, the multi-dose container is adapted to deliver a consistent dose of about 30 μL as a drop of ophthalmic topical solution each time it is used.

With focus on the BSF vial, in some embodiments, this technology relates to a single-use blow-fill-seal (BFS) vial comprising an ophthalmic topical composition comprising about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive. In some embodiments, this technology relates to a single-use blow-fill-seal (BFS) vial comprising an ophthalmic topical composition consisting essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive.

In some embodiments, the single-use blow-fill-seal (BFS) vial comprises or consists essentially of about 1% bevemipretide, about 5.3 mg/mL NaCl, and about 4.16 mg/mL NaH₂PO₄·H₂O, wherein the pH is about 5.8. In some embodiments, the single-use blow-fill-seal (BFS) vial comprises or consists essentially of about 2% bevemipretide, about 3.4 mg/mL NaCl, and about 4.16 mg/mL NaH₂PO₄·H₂O, wherein the pH is about 5.8. In some embodiments, the single-use blow-fill-seal (BFS) vial comprises or consists essentially of about 3% bevemipretide, about 1.5 mg/mL NaCl, and about 4.16 mg/mL NaH₂PO₄·H₂O, wherein the pH is about 5.8.

In some embodiments, the volume of the single-use blow-fill-seal vial is in the range of 0.5 mL to 1.0 mL, inclusive. In some embodiments, the target volume of the single-use blow-fill-seal vial is about 0.8 mL. In some embodiments, the volume of the blow-fill-seal vial is in the range of 0.24 mL to 0.40 mL, inclusive. In some embodiments, the target volume of the composition in the blow-fill-seal vial is about 0.32 mL.

In some embodiments, the composition of the single-use blow-fill-seal vial is sterile. In some embodiments, the composition of the single-use blow-fill-seal vial is preservative-free. In some embodiments, the composition of the single-use blow-fill-seal vial is sterile and preservative-free.

With focus on the multi-dose container, in some embodiments this technology relates to a multi-dose container comprising an ophthalmic topical composition comprising about 1% to about 3% bevemipretide or a pharmaceutically acceptable salt thereof, wherein the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive. In some embodiments, the multi-dose container comprises an ophthalmic topical composition consisting essential of about 1% to about 3% bevemipretide or a pharmaceutically acceptable salt thereof, wherein the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive.

In some embodiments, the multi-dose container comprises or consists essentially of about 1% bevemipretide, about 5.3 mg/mL NaCl, and about 4.16 mg/mL NaH₂PO₄·H₂O, wherein the pH is about 5.8. In some embodiments, the multi-dose container comprises or consists essentially of about 2% bevemipretide, about 3.4 mg/mL NaCl, and about 4.16 mg/mL NaH₂PO₄·H₂O, wherein the pH is about 5.8. In some embodiments, the multi-dose container comprises or consists essentially of about 3% bevemipretide, about 1.5 mg/mL NaCl, and about 4.16 mg/mL NaH₂PO₄·H₂O, wherein the pH is about 5.8.

In some embodiments, the composition of the multi-dose container is sterile. In some embodiments, the composition of the multi-dose container is preservative-free. In some embodiments, the composition of the multi-dose container is sterile and preservative-free.

In some embodiments, the multi-dose container is adapted to maintain sterility of the composition for a week or more, a month or more, three months or more, 6 months or more or a year or more. In some embodiments, the multi-dose container is adapted to comprises between 2.5 mL and 30 mL, inclusive, of ophthalmic topical composition. In some embodiments, the multi-dose container is adapted to deliver a consistent dose of between 25 μL and 35 μL, inclusive, as a drop of ophthalmic topical solution each time it is used. In some embodiments, the multi-dose container is adapted to deliver a consistent dose of about 30 μL as a drop of ophthalmic topical solution each time it is used.

In some embodiments, this technology is directed to a method of preparing a preservative-free composition comprising bevemipretide or a pharmaceutically acceptable salt thereof, the method comprising dissolving in an aqueous medium about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl; and about 4.1 mg/mL to about 4.2 mg/mL NaH₂PO₄·H₂O; and adding sodium hydroxide or hydrochloric acid in amount sufficient to adjust pH of the ophthalmic topical composition to the range of 5.7 to 5.9, inclusive.

In some embodiments of the method, the composition comprises about 1% bevemipretide, about 5.3 mg/mL NaCl; and about 4.16 mg/mL NaH₂PO₄·H₂O; wherein the pH is about 5.8. In some embodiments of the method, the composition comprises about 2% bevemipretide, about 3.4 mg/mL NaCl; and about 4.16 mg/mL NaH₂PO₄·H₂O; wherein the pH is about 5.8. In some embodiments of the method, the composition comprises about 3% bevemipretide, about 1.5 mg/mL NaCl; and about 4.16 mg/mL NaH₂PO₄·H₂O; wherein the pH is about 5.8.

In some embodiments of the method, the composition is sterile.

In some embodiments of the method, after preparation a portion of the sterile composition is transferred to a blow-fill-seal (BFS) vial. In some embodiments of the method, the blow-fill-seal vial is adapted for single-use. In some embodiments, the blow-fill-seal vial is adapted for multi-day/multidose use. In some embodiments of the method, the volume of the single-use blow-fill-seal vial is in the range of 0.5 mL to 1.0 mL, inclusive. In some embodiments of the method, the volume of the blow-fill-seal vial is about 0.8 mL. In some embodiments of the method, the volume of the composition transferred to the blow-fill-seal vial is in the range of 0.24 mL to 0.40 mL, inclusive. In some embodiments of the method, the volume of the composition transferred to the blow-fill-seal vial is about 0.32 mL.

In some embodiments of the method, after preparation a portion of the sterile composition is transferred to a multi-dose container. In some embodiments, the multi-dose container is adapted to maintain sterility of the composition for a week or more, a month or more, three months or more, 6 months or more or a year or more. In some embodiments, the multi-dose container is adapted to comprises between 2.5 mL and 30 mL, inclusive, of ophthalmic topical composition. In some embodiments, the multi-dose container is adapted to deliver a consistent dose of between 25 μL and 35 μL, inclusive, as a drop of ophthalmic topical solution each time it is used. In some embodiments, the multi-dose container is adapted to deliver a consistent dose of about 30 μL as a drop of ophthalmic topical solution each time it is used.

The ophthalmic topical solutions described herein can be administered topically (such as via eye drops) to address ophthalmic medical indications including methods for dosing said ophthalmic topical solutions to address such ophthalmic medical indications. Thus, in some embodiments, this technology is directed to a method of dosing bevemipretide, or a pharmaceutically acceptable salt thereof, to a subject in need thereof, the method comprising administering a preservative-free ophthalmic topical composition once daily, twice daily, or three times daily to the subject wherein the ophthalmic topical composition comprises about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O; and water, wherein pH of the ophthalmic topical composition is in a range of 5.7 to 5.9, inclusive. In some embodiments, the subject has been diagnosed with macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), or oxygen-induced retinopathy. In some embodiments, the subject has been diagnosed with macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy. In some embodiments, the subject has been diagnosed with dry age-related macular degeneration, glaucoma, pigmentary retinopathy, retinitis pigmentosa, diabetic retinopathy, diabetic macular edema, Stargardt's disease, or oxygen-induced retinopathy.

Thus, in some embodiments, this technology is directed to a method of dosing bevemipretide, or a pharmaceutically acceptable salt thereof, to a subject in need thereof, the method comprising administering an ophthalmic topical composition once daily, twice daily, or three times daily to a subject wherein the ophthalmic topical composition consists essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O; and water, wherein pH of the ophthalmic topical composition is in a range of 5.7 to 5.9, inclusive. In some embodiments, the subject has been diagnosed with macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), or oxygen-induced retinopathy. In some embodiments, the subject has been diagnosed with macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy. In some embodiments, the subject has been diagnosed with dry age-related macular degeneration, glaucoma, pigmentary retinopathy, retinitis pigmentosa, diabetic retinopathy, diabetic macular edema, Stargardt's disease, or oxygen-induced retinopathy.

In some embodiments, this technology is directed to a method for treating, preventing, inhibiting, ameliorating, or delaying the onset of dry age-related macular degeneration in a subject in need thereof, the method comprising administering a preservative-free composition to the subject at least once daily, wherein the composition comprises about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein pH of the ophthalmic topical composition is in the range of 5.7 to 5.9, inclusive. In some embodiments, the administering is conducted once daily, twice daily, or three times daily. In some embodiments, this technology is directed to a method for treating, preventing, inhibiting, ameliorating, or delaying the onset of dry age-related macular degeneration in a subject in need thereof, the method comprising administering a composition to the subject at least once daily, wherein the composition consists essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein pH of the ophthalmic topical composition is in the range of 5.7 to 5.9, inclusive. In some embodiments, the administering is conducted once daily, twice daily, or three times daily.

In some embodiments of any of the foregoing dosing or therapeutic methods, the ophthalmic topical composition can be administered using a blow-fill-seal (BFS) vial. In some embodiments, the blow-fill-seal (BFS) vial is adapted for single-use.

In some embodiments of any of the foregoing dosing or therapeutic methods, the ophthalmic topical composition is administered using a multi-dose container. In some embodiments, the multi-dose container comprises between 2.5 mL and 30 mL, inclusive, of ophthalmic topical composition.

In some embodiments of any of the foregoing dosing or therapeutic methods, the ophthalmic topical composition can be administered to one eye of the subject. In some embodiments of any of the foregoing dosing or therapeutic methods, the ophthalmic topical composition is administered to both eyes of the subject. In some embodiments of any of the foregoing dosing or therapeutic methods, the ophthalmic topical composition can be administered at a rate of one drop per eye of the subject per administration. In some embodiments of any of the foregoing dosing or therapeutic methods, the ophthalmic topical composition can be administered at a rate of two or more drops per eye of the subject per administration. In some embodiments of any of the foregoing dosing or therapeutic methods, each drop administered to the eye is in the range of 22 μL to 38 μL, inclusive, in volume. In some embodiments, each drop is about 30 μL in volume. In some embodiments of any of the foregoing dosing or therapeutic methods, the administering is repeated daily for at least 12 weeks, at least 24 weeks, at least 36 weeks, at least 48 weeks, at least 60 weeks, at least 72 weeks, at least 84 weeks, or at least 96 weeks.

In some embodiments of any of the foregoing dosing or therapeutic methods, the composition comprises or consists essentially of about 1% bevemipretide, about 5.3 mg/mL NaCl; and about 4.16 mg/mL NaH₂PO₄·H₂O; wherein the pH is about 5.8. In some embodiments of any of the foregoing dosing or therapeutic methods, the composition comprises or consists essentially of about 2% bevemipretide, about 3.4 mg/mL NaCl; and about 4.16 mg/mL NaH₂PO₄·H₂O; wherein the pH is about 5.8. In some embodiments of any of the foregoing dosing or therapeutic methods, the composition comprises or consists essentially of about 3% bevemipretide, about 1.5 mg/mL NaCl; and about 4.16 mg/mL NaH₂PO₄·H₂O; wherein the pH is about 5.8.

In some embodiments of any of the foregoing dosing or therapeutic methods, the subject has been diagnosed with or is at risk for developing dry age-related macular degeneration. In some embodiments, the subject has drusen. In some embodiments, the subject has been diagnosed with or is at risk for developing geographic atrophy (GA). In some embodiments, the subject has been diagnosed with partial ellipsoid zone attenuation of one or more portions/zones of the macula of at least one eye. In some embodiments, the subject has been diagnosed with total ellipsoid zone attenuation of one or more portions/zone of the macula of at least one eye.

In some embodiments of any of the foregoing dosing or therapeutic methods, the composition is administered to the subject once per day. In some embodiments of any of the foregoing dosing or therapeutic methods, the composition is administered to the subject twice per day. In some embodiments of any of the foregoing dosing or therapeutic methods, the composition is administered to the subject three times per day. In some embodiments of any of the foregoing dosing or therapeutic methods, the composition is sterile. In some embodiments of any of the foregoing dosing or therapeutic methods, the composition is sterile and preservative-free. In some embodiments of any of the foregoing dosing or therapeutic methods, the subject is human.

In some embodiments, this technology is directed to a preservative-free ophthalmic topical composition comprising about 1% bevemipretide, or a pharmaceutically acceptable salt thereof, where the percent bevemipretide is based on the free-base weight of the bevemipretide to volume (w/v) of the composition, about 5.3 mg/mL NaCl; and about 4.16 mg/mL NaH₂PO₄·H₂O; wherein the pH is about 5.8. In some embodiments, this technology is directed to a preservative-free ophthalmic topical composition comprising about 2% bevemipretide, or a pharmaceutically acceptable salt thereof, where the percent bevemipretide is based on the free-base weight of the bevemipretide to volume (w/v) of the composition, about 3.4 mg/mL NaCl; and about 4.16 mg/mL NaH₂PO₄·H₂O; wherein the pH is about 5.8. In some embodiments, this technology is directed to a preservative-free ophthalmic topical composition comprising about 3% bevemipretide, or a pharmaceutically acceptable salt thereof, where the percent bevemipretide is based on the free-base weight of the bevemipretide to volume (w/v) of the composition, about 5.3 mg/mL NaCl; and about 4.16 mg/mL NaH₂PO₄·H₂O; wherein the pH is about 5.8. In some embodiments, the aforementioned compositions are sterile.

In some embodiments, this technology is directed to an ophthalmic topical composition consisting essentially of about 1% bevemipretide, or a pharmaceutically acceptable salt thereof, where the percent bevemipretide is based on the free-base weight of the bevemipretide to volume (w/v) of the composition, about 5.3 mg/mL NaCl; and about 4.16 mg/mL NaH₂PO₄·H₂O; wherein the pH is about 5.8. In some embodiments, this technology is directed to an ophthalmic topical composition consisting essentially of about 2% bevemipretide, or a pharmaceutically acceptable salt thereof, where the percent bevemipretide is based on the free-base weight of the bevemipretide to volume (w/v) of the composition, about 3.4 mg/mL NaCl; and about 4.16 mg/mL NaH₂PO₄·H₂O; wherein the pH is about 5.8. In some embodiments, this technology is directed to an ophthalmic topical composition consisting essentially of about 3% bevemipretide, or a pharmaceutically acceptable salt thereof, where the percent bevemipretide is based on the free-base weight of the bevemipretide to volume (w/v) of the composition, about 5.3 mg/mL NaCl; and about 4.16 mg/mL NaH₂PO₄·H₂O; wherein the pH is about 5.8. In some embodiments, the aforementioned compositions are sterile.

In some embodiments, the aforementioned compositions are packaged in a single-use blow-fill-seal vial. In some embodiments, the aforementioned compositions are packaged in a multi-dose container adapted to maintain sterility of the composition for a week or more, a month or more, three months or more, 6 months or more or a year or more. In some embodiments, the osmolality of the aforementioned compositions is in the range of 260-285 mOsm/kg, inclusive.

In some embodiments, this technology is directed to a method of dosing bevemipretide, or a pharmaceutically acceptable salt thereof, to a subject in need thereof, the method comprising administering as an eye drop an ophthalmic topical composition once daily or twice daily to the subject wherein the ophthalmic topical composition comprises about 1% bevemipretide, or a pharmaceutically acceptable salt thereof, where the percent bevemipretide is based on the free-base weight of the bevemipretide to volume (w/v) of the composition, about 5.3 mg/mL NaCl, and about 4.16 mg/mL NaH₂PO₄·H₂O; wherein the pH is about 5.8. In some embodiments, this technology is directed to a method of dosing bevemipretide, or a pharmaceutically acceptable salt thereof, to a subject in need thereof, the method comprising administering as an eye drop an ophthalmic topical composition once daily or twice daily to the subject wherein the ophthalmic topical composition comprises about 2% bevemipretide, or a pharmaceutically acceptable salt thereof, where the percent bevemipretide is based on the free-base weight of the bevemipretide to volume (w/v) of the composition, about 3.4 mg/mL NaCl, and about 4.16 mg/mL NaH₂PO₄·H₂O; wherein the pH is about 5.8.

In some embodiments, this technology is directed to a method of dosing bevemipretide, or a pharmaceutically acceptable salt thereof, to a subject in need thereof, the method comprising administering as an eye drop an ophthalmic topical composition once daily or twice daily to the subject wherein the ophthalmic topical composition comprises about 3% bevemipretide, or a pharmaceutically acceptable salt thereof, where the percent bevemipretide is based on the free-base weight of the bevemipretide to volume (w/v) of the composition, about 1.5 mg/mL NaCl, and about 4.16 mg/mL NaH₂PO₄·H₂O; wherein the pH is about 5.8.

In some embodiments of the aforementioned dosing methods, the composition is sterile and preservative-free. In some embodiments of the aforementioned dosing methods, the dosing is repeated daily for at least 12 weeks, at least 24 weeks, at least 36 weeks, at least 48 weeks, at least 60 weeks, at least 72 weeks, at least 84 weeks, or at least 96 weeks. In some embodiments of the aforementioned dosing methods, the composition is sterile. In some embodiments of the aforementioned dosing methods, the composition is sterile and preservative-free. In some embodiments of the aforementioned dosing methods, the subject is human.

In some embodiments of the aforementioned dosing methods, the ophthalmic topical composition is packaged in a single-use blow-fill-seal vial. In some embodiments of the aforementioned dosing methods, the ophthalmic topical composition is packaged in a multi-dose container adapted to maintain sterility of the composition for a week or more, a month or more, three months or more, 6 months or more, or a year or more.

In one aspect, the disclosure of the present technology provides a method of dosing (R)-2-amino-N-((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide (bevemipretide), or a pharmaceutically acceptable salt, stereoisomer, tautomer, hydrate, and/or solvate thereof, in a subject in need thereof, the method comprising: (i) administering an ophthalmic topical composition comprising 1% (wt./vol.) bevemipretide, or a pharmaceutically acceptable salt, stereoisomer, tautomer, hydrate, and/or solvate thereof, once daily, twice daily, or three times daily to the subject; (ii) administering an ophthalmic topical composition comprising 2% (wt./vol.) bevemipretide, or a pharmaceutically acceptable salt, stereoisomer, tautomer, hydrate, and/or solvate thereof, once daily, twice daily, or three times daily to the subject; or (iii) administering an ophthalmic topical composition comprising 3% (wt./vol.) bevemipretide, or a pharmaceutically acceptable salt, stereoisomer, tautomer, hydrate, and/or solvate thereof, once daily, twice daily, or three times daily to the subject.

In one aspect, the disclosure of the present technology provides a method for treating, preventing, inhibiting, ameliorating, or delaying the onset of dry age-related macular degeneration, the method comprising dosing (R)-2-amino-N-((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide (bevemipretide), or a pharmaceutically acceptable salt, stereoisomer, tautomer, hydrate, and/or solvate thereof, in a subject in need thereof, the method comprising: (i) administering an ophthalmic topical composition comprising 1% (wt./vol.) bevemipretide, or a pharmaceutically acceptable salt, stereoisomer, tautomer, hydrate, and/or solvate thereof, once daily, twice daily, or three times daily to the subject; (ii) administering an ophthalmic topical composition comprising 2% (wt./vol.) bevemipretide, or a pharmaceutically acceptable salt, stereoisomer, tautomer, hydrate, and/or solvate thereof, once daily, twice daily, or three times daily to the subject; or (iii) administering an ophthalmic topical composition comprising 3% (wt./vol.) bevemipretide, or a pharmaceutically acceptable salt, stereoisomer, tautomer, hydrate, and/or solvate thereof, once daily, twice daily, or three times daily to the subject.

In some embodiments, the subject has been diagnosed with or is at risk for developing dry age-related macular degeneration. In some embodiments, the subject has drusen. In some embodiments, the subject has been diagnosed with or is at risk for developing geographic atrophy (GA). In some embodiments, the ophthalmic topical composition is an eye drop. In some embodiments, the ophthalmic topical composition is a buffered sterile solution. In some embodiments, the subject is human. In some embodiments, the dosing is repeated daily for at least 12 weeks, at least 24 weeks, at least 36 weeks, at least 48 weeks, at least 60 weeks, at least 72 weeks, at least 84 weeks or at least 96 weeks.

### BRIEF DESCRIPTION OF THE DRAWINGS

FIGS. 1A-1C are a series of schematics depicting structure and function of mitochondria present in healthy subjects and unhealthy subjects with dry AMD. FIG. 1A is a series of schematics depicting structure and function of mitochondria present in a healthy subject with an illustration of a healthy mitochondria cristae on the right. FIG. 1B is a series of schematics depicting structure and function of mitochondria present in a subject with dry AMD with an illustration of an unhealthy mitochondria cristae on the right; curvature of the cristae is relaxed leading to separation between complexes of the electron transport chain (ETC), inefficient adenosine triphosphate (ATP) production and resulting generation of reactive oxygen species (ROS). Without intending to be bound to any theory, FIG. 1C is an illustration of a mitochondria cristae of a subject with dry AMD treated with bevemipretide-curvature of the cristae returns thereby restoring efficient electron transport chain activity and normalization of ATP and ROS production.

FIGS. 2A-2B are graphs showing results from the 91-day GLP study demonstrating bevemipretide concentrations in ocular target tissues and other ocular tissues when administered as an ophthalmic topical composition comprising: (i) 1% (wt./vol.) bevemipretide TID (1.2 mg/eye/day); (ii) 2% (wt./vol.) bevemipretide TID (2.4 mg/eye/day); or (iii) 4% (wt./vol.) bevemipretide TID (4.8 mg/eye/day); and showing the concentration of bevemipretide in ocular tissues from the 4% (wt./vol.) TID treatment group after a 4-week (28-day) recovery period. BEVE, bevemipretide; LLOQ, lower limits of quantification; RPE, retinal pigment epithelium; TID, three times daily. * N=6 or 4 (recovery). Missing values were below the LLOQ. Liquid sample LLOQ=0.1 ng/ml; tissue LLOQ=1 or 2 ng/g.

FIGS. 3A-3B are graphs showing the plasma bevemipretide concentrations in the 91-day GLP study in minipigs over time at Day 1 post third dose (FIG. 3A) and at Day 91 post third dose (FIG. 3B) in the following treatment groups: (i) 1% bevemipretide TID (1.2. mg/eye/day); (ii) 2% bevemipretide TID (2.4 mg/eye/day); and (iii) 4% bevemipretide (4.8 mg/eye/day). BEVE, bevemipretide; GSD, geometric standard deviation; TID, three times daily.

FIG. 4 is a process diagram for the manufacture of bevemipretide ophthalmic topical solution and associated packaging the solution into a sterile, blow-fill-seal (BFS) vial suitable for single-use administration to a subject.

FIG. 5 is drawing of the container design of the BFS vial, individually, and in the 5-vial card produced for the clinical trial described herein according to the manufacturing process described in FIG. 4.

FIG. 6A is graphic summary of Part A-single ascending dose (SAD) human clinical study described in Example 2. FIG. 6B is graphic summary of Part B-multiple ascending dose (MAD) human clinical study described in Example 2.

FIG. 7 is a poster providing an overview of the development of topical bevemipretide eye drops for dry AMD.

FIG. 8 is a poster providing an overview of a phase 1 dose escalation study to evaluate safety, tolerability, and pharmacokinetics of bevemipretide eye drops for dry AMD.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, NY, 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

### I. DEFINITIONS

The definitions of certain terms as used in this specification are provided below. Unless defined otherwise, all technical and scientific terms used herein generally have the same meaning as commonly understood by one of ordinary skill in the art to which the present technology belongs.

As used in this specification and the appended claims, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value. With respect to components (e.g., chemical reagents/solvents) of a composition used as a therapeutic agent/composition (e.g., ophthalmic topical composition), the term “about” refers to the variation in amounts that are within the manufacturing specifications for the therapeutic agent/composition as approved by applicable government regulatory bodies (e.g., the Food and Drug Administration (FDA). For example, the phrase “about 1% bevemipretide” as used herein refers to the amount of bevemipretide added to prepare a 1% bevemipretide ophthalmic topical solution for use as a therapeutic agent for human use, where, for example, the specification might state that the concentration (w/v) can vary between 0.95% and 1.05% bevemipretide.

As used herein, the “administrating” or “administration” of an agent (i.e., a therapeutic agent such as bevemipretide) or compound/drug product (including a composition (i.e., a formulation or medicament) comprising bevemipretide) to a subject includes any route of introducing or delivering to a subject a compound, composition formulation or drug product to perform its intended function. Administration can be carried out by any suitable route, such as oral administration. Administration can be carried out subcutaneously. Administration can be carried out topically. Administration can be carried out intraocularly (intravitreally). Administration can be carried out ophthalmically. Administration can be carried out systemically. Administration may be carried out intranasally. Administration can be carried out intravenously. Administration can be carried out intraperitoneally. Administration can be carried out intradermally. Administration can be carried out intrathecally. Administration can be carried out intracerebroventricularly. Administration can be carried out iontophoretically. Administration can be carried out transmucosally. Administration can be carried out intravitreally. Administration can be carried out intramuscularly. Administration includes self-administration, administration by another or administration by a device (e.g., a pump).

As used herein, to “ameliorate” or “ameliorating” a disease or disorder refers to results that, in a statistical sample or specific subject, make the occurrence of the disease, or disorder (or a sign, symptom or condition thereof) better or more tolerable in a sample or subject administered one or more therapeutic agents relative to a control sample, control subject or group of control subjects not administered the therapeutic agent(s).

As used herein, “bevemipretide” (also known as SBT-272) refers to a peptidomimetic having a name: (R)-2-amino-N-((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide, having the chemical abstract service number CAS #: 2356106-71-1), and structure:

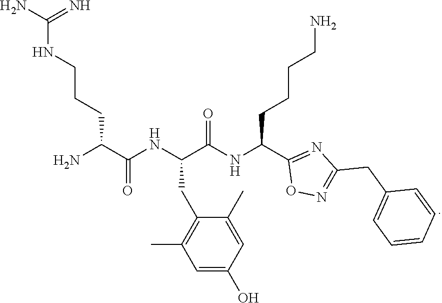 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

For simplicity of use herein (including when used in the claims), “bevemipretide” also refers to and includes all salts forms thereof (e.g., the tris-HCl salt of formula:

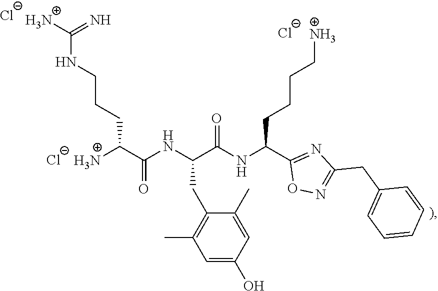 [SMILES: [Cl-] ; Cc1cc(O)cc(C)c1C[C@H](NC(=O)[C@H]([NH3+])CCCNC(=N)[NH3+])C(=O)N[C@@H](CCCC[NH3+])c1nc(Cc2ccccc2)no1 ; [Cl-] ; [Cl-]]

as well as all stereoisomers, tautomers, hydrates, solvates, formulations, and any mixture of forms or combinations of any or all of the forgoing. Similarly, use of the phrase “bevemipretide, or a pharmaceutically acceptable salt thereof in the specification or claims is intended to encompass bevemipretide as well as all pharmaceutically acceptable salts, stereoisomers, tautomers, hydrates, solvates, formulations, and any mixture of forms or combinations of any or all of the forgoing. In some embodiments, one or more of the hydrogen atoms of bevemipretide can be substituted with a deuterium atom or fluoride atom without any significant deviation from its efficacy or therapeutic profile. In such cases, use of the term “bevemipretide” or the phrase “bevemipretide, or a pharmaceutically acceptable salt thereof” in this application and claims is also intended to be inclusive of such deuterium and/or fluorine substituted forms of bevemipretide.

As used herein, the phrase “delaying the onset of” refers to, in a statistical sample, postponing, hindering the occurrence of a disease, or disorder, or causing one or more signs, symptoms or conditions of the disease or disorder to occur more slowly than normal, in a sample or subject administered one or more therapeutic agent(s) relative to a control sample, control subject or group of control subjects not administered the therapeutic agent(s).

As used herein, the term “effective amount” refers to a quantity of a compound, drug product, or composition (including a formulation or medicament) sufficient to achieve a desired therapeutic and/or prophylactic effect, e.g., an amount that treats, prevents, inhibits, ameliorates, or delays the onset of the disease, disorder or condition, or the physiological signs or symptoms of the disease or disorder (or its related signs, symptoms or conditions. In the context of therapeutic or prophylactic applications, in some embodiments, the amount of a composition administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional therapeutic agent(s) could be administered simultaneously, sequentially or by separate administration). The present technology relates to the administration of bevemipretide, optionally with co-administration of one or more additional therapeutic agents . . .

As used herein, the term “hydrate” refers to a compound which is associated (e.g., complexed) with water. The number of the water molecules contained in a hydrate of a compound may be (or may not be) in a definite ratio to the number of the compound molecules in the hydrate.

As used herein, “inhibit” or “inhibiting” refers to the reduction in a sign, symptom, or condition (e.g., risk factor) associated with a disease or disorder causing it to occur more slowly than normal in a sample or subject administered a therapeutic agent(s) relative to a control sample, control subject or control group of subjects not administered the therapeutic agent(s). In one embodiment, inhibit or inhibiting refers to the reduction by at least a statistically significant amount compared to a control sample, control subject or group of control subjects. In one embodiment, inhibit or inhibiting refers to a reduction by at least 5 percent compared to control sample, control subject or group of control subjects. In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control sample, control subject or group of control subjects.

As used herein, “multi-dose container” refers to a container designed to deliver a formulation safely over several days, a week or more, a month or more, 3 months or more, 6 months or more, or a year or more of repeated use. In some embodiments, the “multi-dose container” is adapted to contain and deliver the formulation whilst maintaining sterility of the formulation. In some embodiments, the “multi-dose container” is adapted to deliver eye drops. In some embodiments, the “multi-dose container” is adapted to deliver sterile, preservative-free eye drops over the period of days, weeks, months or years. In some embodiments, the “multi-dose container” is adapted to deliver a consistent dose of between 25 μL and 35 μL, inclusive, as a drop each time it is used. In some embodiments, the “multi-dose container” is adapted to deliver a consistent dose of about 30 μL as a drop each time it is used.

As used herein, “NaH₂PO₄·H₂O” refers to sodium phosphate monobasic monohydrate having a CAS number 10049-21-5 and molecular weight of 137.99 g/mol. Furthermore, it is to be understood that in formulating any of the bevemipretide ophthalmic topical solutions described herein (e.g., the 1%, 2% or 3% bevemipretide solutions), the sodium phosphate monobasic monohydrate can be substituted with other forms of sodium phosphate (for example with sodium phosphate monobasic dihydrate), so long as the weight of reagent used in formulation is adjusted so that the end concentration of sodium phosphate is maintained in accordance with the specifications for the bevemipretide ophthalmic topical solution as if sodium phosphate monobasic monohydrate had been used. In this respect, the other forms of sodium phosphate are equivalents.

The terms “pharmaceutically acceptable carrier” and “carrier” as used herein refer to a diluent, adjuvant, excipient, or vehicle with which a compound/drug product/composition (including a formulation or medicament) is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, “preservative-free” refers to the condition where no preservative is intentionally added to maintain the sterility of a composition described herein. For example, in any embodiment herein including a composition (e.g., an ophthalmic topical composition) that is preservative-free, the composition may not include either of a quaternary ammonium preservative or an oxidizing agent preservative; in any embodiment herein including a composition (e.g., an ophthalmic topical composition) that is preservative-free, the composition may not include benzalkonium chloride, benzododecinium bromide, polyquaternium-1, sodium perborate, and a stabilized oxochloro complex (e.g., a mixture of chlorite, chlorate, and chlorine dioxide).

As used herein, “prevention” or “preventing” of a disease, or disorder refers to results that, in a statistical sample, exhibit a reduction in the occurrence of the disease or disorder (or a sign, symptom or condition thereof) in a sample or subject administered a therapeutic agent(s) relative to a control sample, control subject or group of control subjects not administered the therapeutic agent(s)

As used herein, the phrase “single-use” refers to a drug, composition, medication, or drug product intended for one-time use on a single patient and as part of a single process (e.g., dosing of (e.g., administering to) one or both eyes of a subject with one or more eye drops from the same BFS vial during a dosing event) before disposal.

As used herein, the term “solvate” refers to forms of a compound (e.g., peptide orpeptidomimetic) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, “sterile” refers to a pharmaceutical preparation (e.g., composition, formulation or drug product) that is substantially free of viable microorganisms and bacterial endotoxins in accordance with the specifications for the pharmaceutical preparation as approved by an applicable government regulatory agency (e.g., the FDA). For example, in some embodiments, “sterile” refers to a pharmaceutical preparation meeting the requirements of USP Chapter <71>. In some embodiments, the pharmaceutical preparations meet the requirements of USP Chapter <85> “Bacterial endotoxin test.”

As used herein, the terms “subject” and “patient” are used interchangeably and each refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

It is also to be appreciated that the various modes of treatment or prevention of medical conditions as described herein, in some embodiments, are intended to mean “substantial,” which includes total but also less than total treatment or prevention, and wherein some biologically or medically relevant result is achieved.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the terms “treat,” “treats,” “treating,” or “treatment” refer to therapeutic treatment, wherein the object is to reduce, alleviate or slow down (lessen) the progression a pre-existing disease or disorder (or a sign, symptom or condition thereof). By way of example, but not by way of limitation, a subject is successfully “treated” for a disease if, after receiving an effective amount of the compound/composition/drug product or a pharmaceutically acceptable salt, stereoisomer, tautomer, hydrate, and/or solvate thereof, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder as well as “partial,” where some biologically or medically relevant result is achieved.

As used herein, “water for injection” refers to pharmaceutical grade, highly purified, sterile, and non-pyrogenic water used as a solvent or diluent for pharmaceutical manufacturing.

As used herein, the abbreviation “(wt./vol.)” refers to a ratio of weight of a compound (such as bevemipretide (as a free-base)) to volume of the solution/composition. Instead of the abbreviation “(wt./vol.)”, the abbreviation “(w/v)” can be used. The abbreviation “(wt./vol.)” and “(w/v)” are interchangeable.

### II. Compositions

In some embodiments, the present technology relates to compositions (sometimes referred to as formulations or medicaments) such as solutions/mixtures comprising bevemipretide, or pharmaceutically acceptable salts, stereoisomers, mixtures of stereoisomers, tautomers, hydrates, and/or solvates, and any mixture of forms or combinations of any or all of the foregoing thereof. For example, such compositions (e.g., a pharmaceutical composition or solution) can be formulated as an ophthalmic topical composition suitable for use as an eye drop. Such ophthalmic topical compositions can be used in methods and related uses for addressing ophthalmic disorders and diseases (or signs, symptoms or conditions associated therewith). Such ophthalmic disorders and diseases can be referred to as medical indications. One such medical indication that such ophthalmic topical compositions may be suitable for addressing is dry age-related macular degeneration (i.e., dry AMD).

In some embodiments, said compositions can contain other components such as a tonicity agent, a buffer and solvent. In some embodiments, said compositions are sterile. In some embodiments, said compositions are preservative-free. In some embodiments, said compositions are sterile and preservative-free. In some embodiments, said compositions are adapted for single-use. In some embodiments, said compositions are sterile, preservative-free, and adapted for single-use.

Thus, in some embodiments, this technology is directed to an ophthalmic topical composition comprising about 1% to about 3% bevemipretide or a pharmaceutically acceptable salt thereof, where the stated “percent bevemipretide” is based on the free-base weight of the bevemipretide to volume (w/v) of the composition, about 1.4 mg/mL to about 5.4 mg/mL sodium chloride (NaCl), about 4.1 mg/mL to about 4.2 mg/mL sodium phosphate monobasic monohydrate (NaH₂PO₄·H₂O), and water (generally-water for injection); wherein a pH of the ophthalmic topical composition is in the range of 5.7 to 5.9, inclusive. In preparation of this composition, generally reagents (e.g., bevemipretide, NaCl, and NaH₂PO₄·H₂O) are dissolved in less than the final volume/mass of water, mixed until everything has been dissolved and evenly distributed throughout. If needed the pH can be adjusted with, for example, hydrochloric acid or sodium hydroxide to achieve the desired pH and then additional water is added until the final volume or mass is achieved. The composition can be prepared volumetrically or gravimetrically.

In some embodiments, the ophthalmic topical composition comprises about 1% bevemipretide, about 5.3 mg/mL NaCl, and about 4.16 mg/mL NaH₂PO₄·H₂O, wherein the pH is about 5.8. In some embodiments, the ophthalmic topical composition comprises about 2% bevemipretide, about 3.4 mg/mL NaCl, and about 4.16 mg/mL NaH₂PO₄·H₂O, wherein the pH is about 5.8. In some embodiments, the ophthalmic topical composition comprises about 3% bevemipretide, about 1.5 mg/mL NaCl, and about 4.16 mg/mL NaH₂PO₄·H₂O, wherein the pH is about 5.8.

In some embodiments, the ophthalmic topical composition is sterile. In some embodiments, the ophthalmic topical composition is preservative-free. In some embodiments, the ophthalmic topical composition is sterile and preservative-free.

Thus, in some embodiments, this technology is directed to an ophthalmic topical composition, the composition comprising an aqueous solution prepared by the dissolution in water (generally water for injection); about 1% to about 3% bevemipretide or a pharmaceutically acceptable salt thereof, where the stated “percent bevemipretide” is based on the free-base weight of the bevemipretide to volume (w/v) of the composition, about 1.4 mg/mL to about 5.4 mg/mL sodium chloride (NaCl), about 4.1 mg/mL to about 4.2 mg/ml sodium phosphate monobasic monohydrate (NaH₂PO₄·H₂O), and sufficient sodium hydroxide and/or hydrochloric acid to maintain a desired pH of the composition; wherein the desired pH of the ophthalmic topical composition is in the range of 5.7 to 5.9, inclusive. In preparation of this composition, generally reagents (e.g., bevemipretide, NaCl, and NaH₂PO₄·H₂O) are dissolved in less than the final volume/mass of water, mixed until everything has been dissolved and evenly distributed throughout. The composition can be prepared volumetrically or gravimetrically since the density of the composition is approximately 1 g/mL, and any slight variations in the reagent values described above would be within the tolerances expected for a pharmaceutical composition without deviation from the approved specifications for the clinical product as approved by the appropriate government regulatory body.

In some embodiments, the ophthalmic topical composition comprises about 1% bevemipretide, about 5.3 mg/mL NaCl, and about 4.16 mg/mL NaH₂PO₄·H₂O, wherein the pH is about 5.8. In some embodiments, the ophthalmic topical composition comprises about 2% bevemipretide, about 3.4 mg/mL NaCl, and about 4.16 mg/mL NaH₂PO₄·H₂O, wherein the pH is about 5.8. In some embodiments, the ophthalmic topical composition comprises about 3% bevemipretide, about 1.5 mg/mL NaCl, and about 4.16 mg/mL NaH₂PO₄·H₂O, wherein the pH is about 5.8.

In some embodiments, the ophthalmic topical composition is sterile. In some embodiments, the ophthalmic topical composition is preservative-free. In some embodiments, the ophthalmic topical composition is sterile and preservative-free.

In some embodiments of the foregoing ophthalmic topical composition, essentially no other reagents or ingredients are required to produce a suitable pharmaceutical product. Accordingly, in some embodiments, this technology is directed to ophthalmic topical composition consisting essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water (generally water for injection), wherein a pH of the ophthalmic topical composition is in the range of 5.7 to 5.9, inclusive. The composition can be prepared volumetrically or gravimetrically.

In some embodiments, the ophthalmic topical composition consists essentially of about 1% bevemipretide, about 5.3 mg/mL NaCl, and about 4.16 mg/mL NaH₂PO₄·H₂O, wherein the pH is about 5.8. In some embodiments, the ophthalmic topical composition consists essentially of about 2% bevemipretide, about 3.4 mg/mL NaCl, and about 4.16 mg/mL NaH₂PO₄·H₂O, wherein the pH is about 5.8. In some embodiments, the ophthalmic topical composition consists essentially of about 3% bevemipretide, about 1.5 mg/mL NaCl, and about 4.16 mg/mL NaH₂PO₄·H₂O, wherein the pH is about 5.8.

In some embodiments, the ophthalmic topical composition is sterile. In some embodiments, the ophthalmic topical composition is preservative-free. In some embodiments, the ophthalmic topical composition is sterile and preservative-free.

In some embodiments this technology is directed to a preservative-free ophthalmic topical composition comprising about 1% bevemipretide, or a pharmaceutically acceptable salt thereof, where the percent bevemipretide is based on the free-base weight of the bevemipretide to volume (w/v) of the composition, about 5.3 mg/mL NaCl, and about 4.16 mg/mL NaH₂PO₄·H₂O; wherein the pH is about 5.8. In some embodiments this technology is directed to a preservative-free ophthalmic topical composition comprising about 2% bevemipretide, or a pharmaceutically acceptable salt thereof, where the percent bevemipretide is based on the free-base weight of the bevemipretide to volume (w/v) of the composition, about 3.4 mg/mL NaCl, and about 4.16 mg/mL NaH₂PO₄·H₂O; wherein the pH is about 5.8. In some embodiments this technology is directed to a preservative-free ophthalmic topical composition comprising about 3% bevemipretide, or a pharmaceutically acceptable salt thereof, where the percent bevemipretide is based on the free-base weight of the bevemipretide to volume (w/v) of the composition, about 1.5 mg/mL NaCl, and about 4.16 mg/mL NaH₂PO₄·H₂O; wherein the pH is about 5.8.

In some embodiments this technology is directed to an ophthalmic topical composition consisting essentially of about 1% bevemipretide, or a pharmaceutically acceptable salt thereof, where the percent bevemipretide is based on the free-base weight of the bevemipretide to volume (w/v) of the composition, about 5.3 mg/mL NaCl, and about 4.16 mg/mL NaH₂PO₄·H₂O; wherein the pH is about 5.8. In some embodiments, this technology is directed to an ophthalmic topical composition consisting essentially of about 2% bevemipretide, or a pharmaceutically acceptable salt thereof, where the percent bevemipretide is based on the free-base weight of the bevemipretide to volume (w/v) of the composition, about 3.4 mg/mL NaCl, and about 4.16 mg/mL NaH₂PO₄·H₂O; wherein the pH is about 5.8. In some embodiments this technology is directed to an ophthalmic topical composition consisting essentially of about 3% bevemipretide, or a pharmaceutically acceptable salt thereof, where the percent bevemipretide is based on the free-base weight of the bevemipretide to volume (w/v) of the composition, about 1.5 mg/mL NaCl, and about 4.16 mg/mL NaH₂PO₄·H₂O; wherein the pH is about 5.8. In some embodiments, the ophthalmic topical composition is preservative-free.

In some embodiments of the aforementioned ophthalmic topical compositions, the composition is packaged in a single-use blow-fill-seal vial. In some embodiments of the aforementioned ophthalmic topical compositions, the composition is packaged in a multi-dose container adapted to maintain sterility of the composition for a week or more, a month or more, three months or more, 6 months or more or a year or more.

In some embodiments of the aforementioned ophthalmic topical compositions, the osmolality of the composition is in the range of 200-400 mOsm/kg, inclusive. In some embodiments of the aforementioned ophthalmic topical compositions, the osmolality of the composition is in the range of 250-300 mOsm/kg, inclusive. In some embodiments of the aforementioned ophthalmic topical compositions, the osmolality of the composition is in the range of 260-285 mOsm/kg, inclusive.

Use of the word “about” in the foregoing description of the ophthalmic topical compositions (and generally whenever used with respect to the description of a composition disclosed herein) is intended to encompass exactly the amount stated (e.g., 1%, 2%, or 3%) as well as a range of values (e.g., 0.95% to 1.05% for 1%) that would be within the tolerances expected in pharmaceutical manufacturing processes and still allow the composition to be within specifications for the pharmaceutical product in accordance with the applicable government regulatory body (e.g., the US Food and Drug Administration (FDA) in the United States). Similarly, use of the word “about” in the phrases: “about 1% bevemipretide”, “about 2% bevemipretide”, “about 3% bevemipretide”, “about 1.4 mg/mL to about 5.4 mg/mL sodium chloride (NaCl)”, “about 5.3 mg/mL NaCl”, “about 3.4 mg/mL NaCl”, “about 1.5 mg/mL NaCl”, “about 4.1 mg/mL to about 4.2 mg/mL sodium phosphate monobasic monohydrate (NaH₂PO₄·H₂O)”, and “about 4.16 mg/mL NaH₂PO₄·H₂O”, are intended to encompass a range of values that would be within the tolerances expected in pharmaceutical manufacturing processes and still allow the composition to be within specifications for the pharmaceutical product in accordance with the laws and regulations of the applicable government regulatory body.

The target pH for the foregoing compositions, as an ophthalmic topical compositions/solutions, is 5.8 but a pH within the range of 5.7 to 5.9, inclusive, is acceptable as slight variations from pH 5.8 are, of course, expected from batch-to-batch.

Any of the foregoing ophthalmic topical compositions/solutions can be adapted for single-use delivery if packaged in suitable container. Any of the foregoing compositions can be adapted for multi-day (i.e., multi-dose) delivery if packaged in suitable container.

### III. Drug Products

Any of the aforementioned compositions can be packaged in a container suitable for administration to, or by, a subject. For example, the aforementioned compositions can be packaged in containers suitable for administration as an eye drop. In some embodiments, such containers can be fashioned for single-use or for multiple-day (i.e., multi-dose containers) use. (Campolo et al., A Review of the Containers Available for Multi-Dose Preservative-Free Eye Drops, Biomed J Sci & Tech Res: 45 (1)-2022, doi.org/10.26717/BJSTR.2022.45.007130). Such containers containing the aforementioned compositions can be classified (at least in the United States by the FDA) as a combination product. In essence, they are a pharmaceutical solution packaged in a dispenser (i.e., a device as classified by the FDA) suitable for administration to, or by, the subject. These are referred to herein as a “drug product” as these can be marketed and sold in this format. Eye drop solutions are often intended to be sterile when dispensed. To maintain sterility over repeated dosing from a container after first opening of the container, a preservative is often added. However, preservatives can irritate the eye of some subjects so it can be desirable to go preservative-free. Maintaining sterility of a preservative-free solution over periods of time and through repeated dispensing can be challenging. However, single-use and multi-dose containers suitable for delivery of preservative-free, sterile solutions/formulations as eye drops are known and commonly used for commercial ophthalmic solutions. Id.

### 1. Blow-Fill-Seal (BFS) Packaging/Containers/Vials

One suitable drug product embodiment is a blow-fill-seal (BFS) container, such as a vial, containing the ophthalmic topical solution. Such a container can be easily manufactured and easily adapted in size, shape and volume for single-use, including single-use administration of an eye drop formulation. Single-use eye drop dispensers are the gold standard for dispensing sterile formulations (Campolo et al., at p. 36038). BFS manufacturing process is very adaptable and widely used in the pharmaceutical industry. Id.

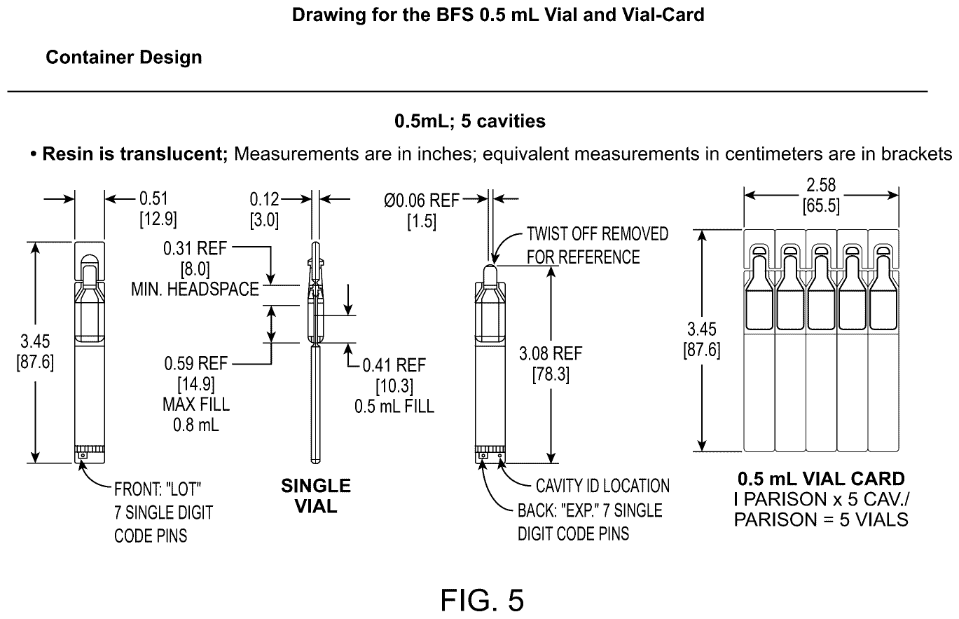

The blow-fill-seal (BFS) manufacturing process functions similarly to conventional extrusion blow molding and typically takes place within an integrated BFS machine. In some embodiments, a plastic polymer resin is heated and compressed, allowing it to be extruded in tubular form and be taken over by an open two-part mold to form the container. The shape, volume and other characteristics/features of the container are therefore dictated by the characteristics of the mold. Then, the mold closes which welds the bottom of the container. Simultaneously, the parison above the mold is cut, or the filling needles are placed in the parison head without the parison being cut (rotary BFS type). Next, a filling mandrel with blowing air function is placed in the neck area that seals the container. Sterile compressed air is then introduced through the filling mandrel to inflate and form the container. After the BFS container has been formed, the desired liquid is filled into the container through the filling mandrel unit. Then, the filling mandrel unit is lifted off, and the head mold hermetically seals the container. Simultaneously, the head contour is formed by vacuum. In the last step, the mold opens and the finished container leaves the mold. An example of a finished product of BFS manufacture can be found at FIG. 5. One process cycle takes a few seconds. The process speed and thus process output largely depends upon the BFS container size and the BFS machinery dimensioning. The output of the Aseptic BFS machines varies based on manufacturer and size. Current Aseptic BFS machines can produce about 4,000 to 35,000 units or containers/hour.

The BFS processes can be made as an aseptic filling process, which produces sterile products. Aseptic BFS machines must be designed in a way that prevents extraneous contamination. Thus, rotary-type BFS machines are placed in classified areas same as shuttle-type BFS machines (open parison), which have a cleanroom shroud grade-A-compliant provided with sterilized air and kept under overpressure. Automatic SIP programs are used to sterilize the BFS equipment and this avoids human interventions. Due to automatic start up and filling processes BFS machines require no human interaction during the actual BFS process. BFS machines are typically fitted with several different sterilizing air filtration systems for the buffer air, support parison air and air shroud of sterile grade A air. The sterile air systems are typically integrated into the BFS apparatus to ensure aseptic operations.

Advantages of BFS manufacturing include custom container design, high output, and low cost. Additionally, the containers are lightweight and shatterproof and therefore more amenable to shipping and storage. The containers can be designed for single-use or multiple day use (though single-use containers are more common for dispensing eye drop formulations)—which can be advantageous for users, such as patients, particularly when traveling, given their lightweight, shatterproof properties, small size, and disposable nature. Finally, the BFS process is more effective at producing sterile products than processes that involve filling of open containers.

### 2. Multi-Dose Packaging/Containers

One suitable drug product embodiment is a multi-dose container suitable for administration of multiple doses of ophthalmic topical solution as an eye drop over days, weeks, months, or years. In some embodiments, the ophthalmic topical solution disclosed herein is preservative-free and/or sterile and can remain sterile through repeated cycles of eye drop dispensing. A review of commercially available container embodiments suitable for maintaining sterility of formulations (including preservative-free formulations) throughout repeated eye drop delivery over days, weeks, months, and years has been published by Campolo et al. (See: Campolo et al., entire document).

Maintaining sterility of the solution in the multi-dose container through the repeated dosing is a challenge because it involves dispensing the liquid solution from the container without contaminating the bulk solution in the container and replacing the dispensed liquid solution with sterile air to equalize the pressure in the container after loss of a volume of liquid. Hence, keeping the solution in the container sterile over repeated dosing requires sophisticated packaging to maintain product integrity. Campolo et al. is a review article discussing various commercially available container embodiments which are typically fitted with a specially adapted cap suitable for dispensing the liquid solution in the container as an eye drop, whilst maintaining sterility of the remaining solution in the container over days, weeks, months, or even years. One such embodiment is the Aptar Ophthalmic Squeeze Dispenser as described in the Aptar Product Brochure-published 3/2026 (available at: aptar.com/en-us/products/pharmaceutical-ophthalmic-squeeze-dispenser-technology-platform). Several other embodiments are discussed by Campolo et al; any of which would be a suitable embodiment of a multi-dose container as disclosed herein.

### 3. Some Embodiments of Drug Products (i.e., Ophthalmic Topical Solution Packaged in a Suitable Dispensing Device)

Thus, in some embodiments, this technology relates to a packaged ophthalmic topical composition comprising a blow-fill-seal (BFS) vial, wherein the blow-fill-seal vial comprises a composition comprising about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl; about 4.1 mg/mL to about 4.2 mg/mL NaH₂PO₄·H₂O, and water, wherein a pH of the ophthalmic topical composition is in the range of 5.7 to 5.9, inclusive. A suitable BFS vial configuration is illustrated in FIG. 5.

In some embodiments, the packaged ophthalmic topical composition comprises about 1% bevemipretide, about 5.3 mg/mL NaCl, and about 4.16 mg/mL NaH₂PO₄·H₂O, wherein the pH is about 5.8. In some embodiments, the packaged ophthalmic topical composition comprises about 2% bevemipretide, about 3.4 mg/mL NaCl, and about 4.16 mg/mL NaH₂PO₄·H₂O, wherein the pH is about 5.8. In some embodiments, the packaged ophthalmic topical composition comprises about 3% bevemipretide, 1.5 mg/mL NaCl, and about 4.16 mg/mL NaH₂PO₄·H₂O, wherein the pH is about 5.8.

As noted previously, the characteristics/features of the BFS vial can be controlled by design of the mold used to fashion the container from the extruded resin. Thus, in some embodiments, the volume of the blow-fill-seal vial of the packaged ophthalmic topical composition is in the range of 0.5 mL to 1.0 mL, inclusive. In some embodiments, the target volume of the blow-fill-seal vial of the packaged ophthalmic topical composition is about 0.8 mL.

The volume of liquid (e.g., ophthalmic topical composition) sited in (e.g., injected into) the BFS vial during the manufacturing process can be controlled by the equipment design and adjustment of various parameters of machine operation, all of which is well-understood and easily controlled. Thus, in some embodiments, the volume of the composition in the blow-fill-seal vial of the packaged ophthalmic topical composition is in the range of 0.24 mL to 0.40 mL, inclusive. In some embodiments, the target volume of the composition in the blow-fill-seal vial of the packaged ophthalmic topical composition is about 0.32 mL.

The mold used to fashion the container in the BFS equipment can be designed to create and fill a multiple vial article (such as a multi-vial card), thereby creating and filling multiple vials simultaneously. Therefore, in some embodiments, the blow-fill-seal vial of the packaged composition is linked physically to one more additional blow-fill-seal vials also containing the ophthalmic topical composition. For example, the blow-fill-seal vial is linked physically to 4 additional blow-fill-seal vials also containing the ophthalmic topical composition to create a 5-unit article of the packaged ophthalmic topical composition; an illustration of one such embodiment is presented in FIG. 5. In some embodiments, the blow-fill-seal vial is linked physically to 6 additional blow-fill-seal vials also containing the ophthalmic topical composition to create a 7-unit article of the packaged ophthalmic topical composition that would be suitable as a one week (7-day) supply of drug product if dosing/administration occur once per day. In some embodiments, the blow-fill-seal vial is linked physically to 13 additional blow-fill-seal vials also containing the ophthalmic topical composition to create a 14-unit article of the packaged ophthalmic topical composition that would be suitable as a two week (14 day) supply of drug product if dosing/administration occur once per day or a one week (7 day supply) if dosing/administration occur twice per day. In some embodiments, the blow-fill-seal vial is linked physically to 27 additional blow-fill-seal vials also containing the ophthalmic topical composition to create a 28-unit article of the packaged ophthalmic topical composition that would be suitable as a 28-day supply of drug product if dosing/administration occur once per day or a two week (14 day supply) if dosing/administration occur twice per day. In some embodiments, the blow-fill-seal vial is linked physically to 29 or 30 additional blow-fill-seal vials also containing the ophthalmic topical composition to create a 30 or 31-unit article of the packaged ophthalmic topical composition that would be suitable as a one month supply of drug product if dosing/administration occur once per day. In some embodiments, the blow-fill-seal vial is linked physically to 1 to 30 additional blow-fill-seal vials also containing the ophthalmic topical composition to create a 2 to 31 unit article.

In some embodiments, the blow-fill-seal vial of the packaged ophthalmic topical composition comprises low density polyethylene (LDPE). In some embodiments, the blow-fill-seal vial of the packaged ophthalmic topical composition comprises a twist off cap. In some embodiments, the packaged ophthalmic topical composition is sterile. In some embodiments, the packaged ophthalmic topical composition is preservative-free. In some embodiments, the packaged ophthalmic topical composition is sterile and preservative-free. In some embodiments, the packaged ophthalmic topical composition is adapted for single use.

With focus on the BSF vial, in some embodiments, this technology further relates to a single-use blow-fill-seal (BFS) vial comprising an ophthalmic topical composition comprising about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive. In some embodiments, this technology further relates to a single-use blow-fill-seal (BFS) vial comprising an ophthalmic topical composition consisting essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄. H₂O, and water, wherein a pH of the ophthalmic topical composition is in the range of 5.7 to 5.9, inclusive.

In some embodiments, the single-use blow-fill-seal (BFS) vial comprises or consists essentially of about 1% bevemipretide, about 5.3 mg/mL NaCl, and about 4.16 mg/mL NaH₂PO₄·H₂O, wherein the pH is about 5.8. In some embodiments, the single-use blow-fill-seal (BFS) vial comprises or consists essentially of about 2% bevemipretide, about 3.4 mg/mL NaCl, and about 4.16 mg/mL NaH₂PO₄·H₂O, wherein the pH is about 5.8. In some embodiments, the single-use blow-fill-seal (BFS) vial comprises or consists essentially of about 3% bevemipretide, about 1.5 mg/mL NaCl, and about 4.16 mg/mL NaH₂PO₄·H₂O, wherein the pH is about 5.8.

In some embodiments, the volume of the single-use blow-fill-seal vial is in the range of 0.5 mL to 1.0 mL, inclusive. In some embodiments, the target volume of the single-use blow-fill-seal vial is about 0.8 mL. In some embodiments, the volume of the blow-fill-seal vial is in the range of 0.24 mL to 0.40 mL, inclusive. In some embodiments, the target volume of the composition in the blow-fill-seal vial is about 0.32 mL.

In some embodiments, the composition of the single-use blow-fill-seal vial is sterile. In some embodiments, the composition of the single-use blow-fill-seal vial is preservative-free. In some embodiments, the composition of the single-use blow-fill-seal vial is sterile and preservative-free.

As previously discussed, the ophthalmic topical composition can be packaged in a multi-dose container. Advantages of the multi-dose container include less economic and environmental waste associated with manufacturing, shipping, storing, and disposing of numerous single-dose containers. A multi-dose container would likely also be more convenient for the subject/patient to store and transport for repeated dosing/administration. However, a significant drawback to the multi-dose container is maintaining sterility throughout repeated dosing if that is a desired feature of the ophthalmic topical composition-which is often the case. However, multi-dose containers can be adapted to maintain sterility throughout repeated dosing for days, weeks, months, or even years.

Thus, with focus on the multi-dose container, in some embodiments this technology relates to a multi-dose container comprising an ophthalmic topical composition comprising about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive. In some embodiments, the multi-dose container comprises an ophthalmic topical composition consisting essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein a pH of the ophthalmic topical composition is in the range of 5.7 to 5.9, inclusive.

In some embodiments, the multi-dose container comprises or consists essentially of about 1% bevemipretide, about 5.3 mg/mL NaCl, and about 4.16 mg/mL NaH₂PO₄·H₂O, wherein the pH is about 5.8. In some embodiments, the multi-dose container comprises or consists essentially of about 2% bevemipretide, about 3.4 mg/mL NaCl, and about 4.16 mg/mL NaH₂PO₄·H₂O, wherein the pH is about 5.8. In some embodiments, the multi-dose container comprises or consists essentially of about 3% bevemipretide, about 1.5 mg/mL NaCl, and about 4.16 mg/mL NaH₂PO₄·H₂O, wherein the pH is about 5.8.

In some embodiments, the composition of the multi-dose container is sterile. In some embodiments, the composition of the multi-dose container is preservative-free. In some embodiments, the composition of the multi-dose container is sterile and preservative-free.

In some embodiments, the multi-dose container is adapted to maintain sterility of the composition for a week or more, a month or more, three months or more, 6 months or more, or a year or more. In some embodiments, the multi-dose container is adapted to comprise between 2.5 mL and 30 mL, inclusive, of ophthalmic topical composition. For example, the multi-dose container can be adapted to hold about 2.5 mL of ophthalmic topical composition, about 5.0 mL of ophthalmic topical composition, about 7.0 mL of ophthalmic topical composition, about 7.5 mL of ophthalmic topical composition, about 10.0 mL of ophthalmic topical composition, about 11.0 mL of ophthalmic topical composition, about 15.0 mL of ophthalmic topical composition, or about 30.0 mL of ophthalmic topical composition. In some embodiments, the multi-dose container can be adapted to comprise between 5.0 mL and 20 mL, inclusive.

In some embodiments, the multi-dose container is adapted to deliver a consistent dose of between 25 μL and 35 μL, inclusive, as a drop of ophthalmic topical solution each time it is used. In some embodiments, the multi-dose container is adapted to deliver a consistent dose of about 30 μL as a drop of ophthalmic topical solution each time it is used.

### IV. Methods of Preparing Compositions

The aforementioned compositions comprising bevemipretide can be prepared using manufacturing processes well-known in the pharmaceutical industry. Indeed, bevemipretide is highly soluble in aqueous solution and readily dissolves in water. For that reason, bevemipretide is well-suited for use in scalable batch processes. Similarly, the other reagent components (e.g., NaCl and NaH₂PO₄·H₂O) of the aforementioned compositions readily dissolve in water at high concentration. Accordingly no special solvents are required and generally only pH need be adjusted to a desired pH for the intended product. pH can be adjusted using commonly used acids and bases such as hydrochloric acid (HCl—to reduce pH) and sodium hydroxide (NaOH—to increase pH). As water is the base solvent for most eye drops, water is well suited as the solvent of choice for the ophthalmic topical compositions described herein.

Thus, in some embodiments, this technology is directed to a method of preparing a preservative-free composition comprising bevemipretide or a pharmaceutically acceptable salt thereof, the method comprising dissolving in an aqueous medium about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl; and about 4.1 mg/mL to about 4.2 mg/mL NaH₂PO₄·H₂O; and adding sodium hydroxide or hydrochloric acid in amount sufficient to adjust pH of the ophthalmic topical composition to the range of 5.7 to 5.9, inclusive.

Thus, in some embodiments, this technology is directed to a method of preparing a composition consisting essentially of (i) bevemipretide or a pharmaceutically acceptable salt thereof, (ii) NaCl, (iii) NaH₂PO₄·H₂O, and (iv) sodium hydroxide and/or hydrochloric acid as needed for pH adjustment, the method comprising dissolving in an aqueous medium about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl; and about 4.1 mg/mL to about 4.2 mg/mL NaH₂PO₄·H₂O; and adding sodium hydroxide or hydrochloric acid in amount sufficient to adjust pH of the ophthalmic topical composition to the range of 5.7 to 5.9, inclusive.

In some embodiments of the method, the composition comprises about 1% bevemipretide, about 5.3 mg/mL NaCl; and about 4.16 mg/mL NaH₂PO₄·H₂O; wherein the pH is about 5.8. In some embodiments of the method, the composition comprises about 2% bevemipretide, about 3.4 mg/mL NaCl; and about 4.16 mg/mL NaH₂PO₄·H₂O; wherein the pH is about 5.8. In some embodiments of the method, the composition comprises about 3% bevemipretide, about 1.5 mg/mL NaCl; and about 4.16 mg/mL NaH₂PO₄·H₂O; wherein the pH is about 5.8.

In some embodiments of the method, the composition consists essentially of about 1% bevemipretide, about 5.3 mg/mL NaCl; and about 4.16 mg/mL NaH₂PO₄·H₂O; wherein the pH is about 5.8 as adjusted by the addition or sodium hydroxide or hydrochloric acid as needed. In some embodiments of the method, the composition consists essentially of about 2% bevemipretide, about 3.4 mg/mL NaCl; and about 4.16 mg/mL NaH₂PO₄·H₂O; wherein the pH is about 5.8 as adjusted by the addition or sodium hydroxide or hydrochloric acid as needed. In some embodiments of the method, the composition consists essentially of about 3% bevemipretide, about 1.5 mg/mL NaCl; and about 4.16 mg/mL NaH₂PO₄·H₂O; wherein the pH is about 5.8 as adjusted by the addition or sodium hydroxide or hydrochloric acid as needed.

In some embodiments of the method, the composition is sterile. In some embodiments of the method, the composition is preservative-free. In some embodiments of the method, the composition is sterile and preservative-free.

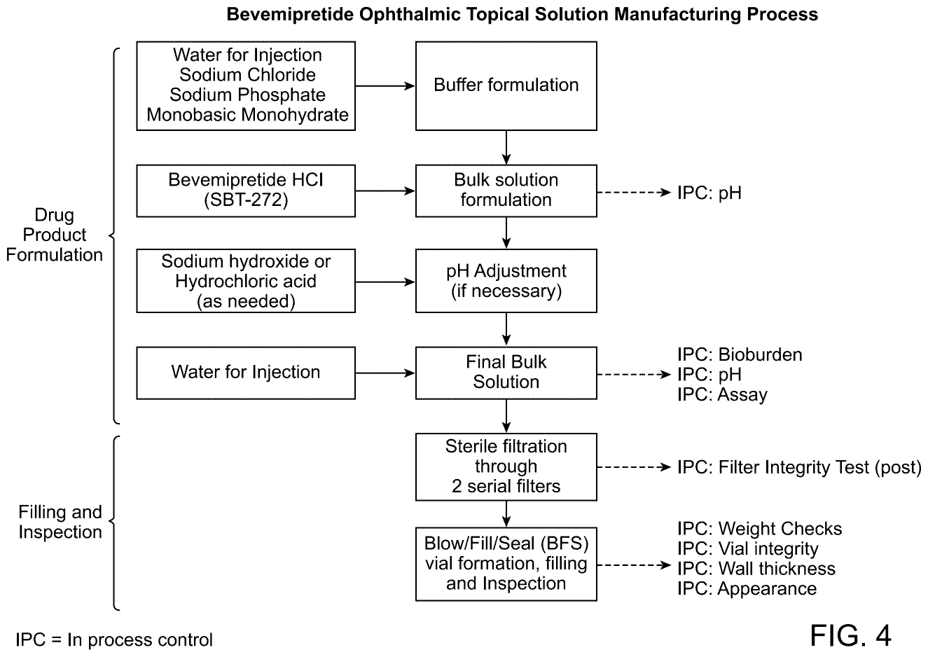

In some embodiments of the method, after preparation a portion of the composition is transferred to a blow-fill-seal (BFS) vial. In some embodiments of the method, the blow-fill-seal vial is adapted for single-use. The composition can be made sterile when being transferred to the blow-fill-seal (BFS) vial, for example, by passing it through a filter adapted to remove bacteria, fungi and particulates that would otherwise contaminate the solution. Such filtration processes are well-known and often used in the pharmaceutical manufacturing industry and was incorporated into the production the BFS vials used in the phase 1 clinical study described below in Example 2 (see also, FIG. 4). In some embodiments, the blow-fill-seal vial is adapted for multi-day/multidose use.

In some embodiments of the method, the volume of the single-use blow-fill-seal vial is in the range of 0.5 mL to 1.0 mL, inclusive. In some embodiments of the method, the volume of the blow-fill-seal vial is about 0.8 mL. In some embodiments of the method, the volume of the composition transferred to the blow-fill-seal vial is in the range of 0.24 mL to 0.40 mL, inclusive. In some embodiments of the method, the volume of the composition transferred to the blow-fill-seal vial is about 0.32 mL.

In some embodiments of the method, after preparation a portion of the composition is transferred to a multi-dose container. The composition can be made sterile, for example, by passing it through a suitably adapted filter as described previously during the transfer. In some embodiments, the multi-dose container is adapted to maintain sterility of the composition for a week or more, a month or more, three months or more, 6 months or more or a year or more. In some embodiments, the multi-dose container is adapted to comprises between 2.5 mL and 30 mL, inclusive, of the composition. In some embodiments, the multi-dose container can be adapted to hold about 2.5 mL of the composition, about 5.0 mL of the composition, about 7.0 mL of the composition, about 7.5 mL of the composition, about 10.0 mL of the composition, about 11.0 mL of the composition, about 15.0 mL of the composition, or about 30.0 mL of the composition. In some embodiments, the multi-dose container can be adapted to comprise between 5.0 mL and 20 mL. As previously described, in some embodiments, the multi-dose container can, after filling, be sealed with a cap that is adapted to dispense drops repeatedly over days, weeks, months or even years whilst maintaining sterility of the ophthalmic topical composition disposed therein if sterile when manufactured. In some embodiments, the multi-dose container is adapted to deliver a consistent dose of about 30 μL as a drop of ophthalmic topical solution each time it is used.

### V. Dosing Methods, Therapeutic Methods and Uses

The compositions (e.g., ophthalmic topical compositions/solutions) and drug products (e.g., packaged ophthalmic topical composition/solutions, single-use blow-fill-seal (BFS) vials, multi-dose BFS vials and multi-dose containers comprising an ophthalmic topical composition/solution) can be used to address various ophthalmic medical indications such as macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), and/or oxygen-induced retinopathy in subjects (e.g., human subjects) in need thereof. In some embodiments, the ophthalmic topical compositions/solutions and drug products disclosed herein are used to address macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy. In some embodiments, the ophthalmic topical compositions/solutions and drug products disclosed herein are used to address dry age-related macular degeneration, glaucoma, pigmentary retinopathy, retinitis pigmentosa, diabetic retinopathy, diabetic macular edema, Stargardt's disease, or oxygen-induced retinopathy. In some embodiments, the ophthalmic topical compositions/solutions and drug products disclosed herein are used to address dry age-related macular degeneration.

In some embodiments, bevemipretide, or a pharmaceutically acceptable salt, stereoisomer, tautomer, hydrate, solvate, or any mixture of forms or combinations of any or all of the foregoing, is dosed to/administered to a subject in need thereof to treat, prevent, inhibit, ameliorate, or delay the onset of any of the aforementioned ophthalmic medical indications, such as age-related macular degeneration (AMD). Subjects having or at risk for AMD can be identified by, e.g., any combination of diagnostic or prognostic assays. The progression of AMD can be determined using methods and techniques known in the art. Age-related macular degeneration can be characterized as either dry age-related macular degeneration or wet age-related macular degeneration.

Dry macular degeneration, which accounts for about 80-90 percent of all cases, is also known as atrophic, nonexudative, or drusenoid macular degeneration. With dry macular degeneration, drusen typically accumulate beneath the retinal pigment epithelium tissue. Vision loss subsequently occurs when drusen interfere with the function of photoreceptors in the macula. Symptoms of dry macular generation include, but are not limited to, distorted vision, center-vision distortion, light or dark distortion, and/or changes in color perception. Dry macular degeneration can result in the gradual loss of vision. Specific damage to the retinal pigmented epithelial (RPE) cells is a hallmark of age-related macular degeneration (AMD), and RPE cell cultures are frequently used as in vitro models of dry AMD.

Thus, in some embodiments, this technology is directed to a method of dosing bevemipretide, or a pharmaceutically acceptable salt thereof, to a subject in need thereof, the method comprising: administering a preservative-free ophthalmic topical composition once daily, twice daily, or three times daily to the subject, wherein the ophthalmic topical composition comprises about 1% to about 3% bevemipretide or a pharmaceutically acceptable salt thereof, where the percent bevemipretide is based on the free-base weight of the bevemipretide to volume (w/v) of the composition about 1.4 mg/mL to about 5.4 mg/ml NaCl, about 4.1 mg/mL to about 4.2 mg/mL NaH₂PO₄·H₂O, and water, wherein pH of the ophthalmic topical composition is in a range of 5.7 to 5.9, inclusive. In some embodiments of the dosing method, the subject has been diagnosed with macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), or oxygen-induced retinopathy. In some embodiments of the method, the subject has been diagnosed with macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy. In some embodiments, the subject has been diagnosed with dry age-related macular degeneration, glaucoma, pigmentary retinopathy, retinitis pigmentosa, diabetic retinopathy, diabetic macular edema, Stargardt's disease, or oxygen-induced retinopathy. In some embodiments, the subject has been diagnosed with dry age-related macular degeneration.

In some embodiments, this technology is directed to a method of dosing bevemipretide, or a pharmaceutically acceptable salt thereof, to a subject in need thereof, the method comprising administering an ophthalmic topical composition once daily, twice daily, or three times daily to the subject, wherein the ophthalmic topical composition consists essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein pH of the ophthalmic topical composition is in a range of 5.7 to 5.9, inclusive. In some embodiments of the dosing method, the subject has been diagnosed with macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), or oxygen-induced retinopathy. In some embodiments of the method, the subject has been diagnosed with macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy. In some embodiments, the subject has been diagnosed with dry age-related macular degeneration, glaucoma, pigmentary retinopathy, retinitis pigmentosa, diabetic retinopathy, diabetic macular edema, Stargardt's disease, or oxygen-induced retinopathy. In some embodiments, the subject has been diagnosed with dry age-related macular degeneration.

In some embodiments, this technology is directed to a method of dosing bevemipretide, or a pharmaceutically acceptable salt thereof, to a subject in need thereof, the method comprising administering as an eye drop an ophthalmic topical composition once daily or twice daily to the subject wherein the ophthalmic topical composition comprises about 1% bevemipretide, or a pharmaceutically acceptable salt thereof, where the percent bevemipretide is based on the free-base weight of the bevemipretide to volume (w/v) of the composition, about 5.3 mg/mL NaCl, and about 4.16 mg/mL NaH₂PO₄·H₂O; wherein the pH is about 5.8. In some embodiments, this technology is directed to a method of dosing bevemipretide, or a pharmaceutically acceptable salt thereof, to a subject in need thereof, the method comprising administering as an eye drop an ophthalmic topical composition once daily or twice daily to the subject wherein the ophthalmic topical composition comprises about 2% bevemipretide, or a pharmaceutically acceptable salt thereof, where the percent bevemipretide is based on the free-base weight of the bevemipretide to volume (w/v) of the composition, about 3.4 mg/mL NaCl, and about 4.16 mg/mL NaH₂PO₄·H₂O; wherein the pH is about 5.8. In some embodiments, this technology is directed to a method of dosing bevemipretide, or a pharmaceutically acceptable salt thereof, to a subject in need thereof, the method comprising administering as an eye drop an ophthalmic topical composition once daily or twice daily to the subject wherein the ophthalmic topical composition comprises about 3% bevemipretide, or a pharmaceutically acceptable salt thereof, where the percent bevemipretide is based on the free-base weight of the bevemipretide to volume (w/v) of the composition, about 1.5 mg/mL NaCl, and about 4.16 mg/mL NaH₂PO₄·H₂O; wherein the pH is about 5.8.

In some embodiments of the aforementioned dosing methods, the ophthalmic topical composition is sterile. In some embodiments of the aforementioned dosing methods, the ophthalmic topical composition is preservative-free. In some embodiments of the aforementioned dosing methods, the ophthalmic topical composition is sterile and preservative-free. In some embodiments of the aforementioned dosing methods, the ophthalmic topical composition can be packaged in a single-use blow-fill-seal vial. In some embodiments of the aforementioned dosing methods, the ophthalmic topical composition is packaged in a multi-dose container adapted to maintain sterility of the composition for a week or more, a month or more, three months or more, 6 months or more or a year or more.

In some embodiments of the aforementioned dosing methods, the dosing can be repeated daily for at least 12 weeks, at least 24 weeks, at least 36 weeks, at least 48 weeks, at least 60 weeks, at least 72 weeks, at least 84 weeks, or at least 96 weeks. In some embodiments of the aforementioned dosing methods, the subject is human.

In some embodiments, this technology is directed to a method for treating, preventing, inhibiting, ameliorating, or delaying the onset of dry age-related macular degeneration in a subject in need thereof, the method comprising administering a preservative-free composition to the subject at least once daily, wherein the composition comprises about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein pH of the ophthalmic topical composition is in the range of 5.7 to 5.9, inclusive. In some embodiments of the method, the administering is conducted once daily, twice daily, or three times daily.

In some embodiments, this technology is directed to a method for treating, preventing, inhibiting, ameliorating, or delaying the onset of dry age-related macular degeneration in a subject in need thereof, the method comprising administering a composition to the subject at least once daily, the composition consisting essentially of about 1% to about 3% bevemipretide or a pharmaceutically acceptable salt thereof, where the percent bevemipretide is based on the free-base weight of the bevemipretide to volume (w/v) of the composition, about 1.4 mg/mL to about 5.4 mg/mL NaCl, about 4.1 mg/mL to about 4.2 mg/mL NaH₂PO₄·H₂O, and water, wherein pH of the ophthalmic topical composition is in the range of 5.7 to 5.9, inclusive. In some embodiments of the method, the administering is conducted once daily, twice daily, or three times daily.

In some embodiments of the aforementioned dosing methods or therapeutic methods, the ophthalmic topical composition can be administered from/using a blow-fill-seal (BFS) vial. In some embodiments, the blow-fill-seal (BFS) vial is adapted for single-use. In some embodiments, the blow-fill-seal (BFS) vial is adapted for multi-dose-use.

In some embodiments of the aforementioned dosing methods or therapeutic methods, the ophthalmic topical composition can be administered using a multi-dose container. In some embodiments, multi-dose container comprises between 2.5 mL and 30 mL, inclusive, of ophthalmic topical composition. For example, the multi-dose container can be adapted to hold about 2.5 mL of the ophthalmic topical composition, about 5.0 mL of the ophthalmic topical composition, about 7.0 mL of the ophthalmic topical composition, about 7.5 mL of the ophthalmic topical composition, about 10.0 mL of the ophthalmic topical composition, about 11.0 mL of the ophthalmic topical composition, about 15.0 mL of the ophthalmic topical composition, or about 30.0 mL of the ophthalmic topical composition. In some embodiments, the multi-dose container can be adapted to comprise between 5.0 mL and 20 mL.

In some embodiments of the aforementioned dosing methods or therapeutic methods, the ophthalmic topical composition is administered to one eye of the subject. In some embodiments, the ophthalmic topical composition is administered to both eyes of the subject. In some embodiments, the ophthalmic topical composition is administered at a rate of one drop per eye of the subject per administration. In some embodiments, the ophthalmic topical composition is administered at a rate of two or more drops per eye of the subject per administration. In some embodiments, each drop administered to the eye is in the range of 22 μL to 38 μL, inclusive, in volume. In some embodiments, each drop administered to the eye is about 30 μL in volume. In some embodiments, the administering is repeated daily for at least 12 weeks, at least 24 weeks, at least 36 weeks, at least 48 weeks, at least 60 weeks, at least 72 weeks, at least 84 weeks, or at least 96 weeks.

In some embodiments of the aforementioned dosing methods or therapeutic methods, the composition, as appropriate, comprises, or consists essentially of, about 1% bevemipretide, about 5.3 mg/mL NaCl, and about 4.16 mg/mL NaH₂PO₄·H₂O, wherein the pH is about 5.8. In some embodiments, the composition, as appropriate, comprises, or consists essentially of, about 2% bevemipretide, about 3.4 mg/mL NaCl, and about 4.16 mg/mL NaH₂PO₄·H₂O, wherein the pH is about 5.8. In some embodiments, the composition, as appropriate, comprises, or consists essentially of, about 3% bevemipretide, about 1.5 mg/mL NaCl, and about 4.16 mg/mL NaH₂PO₄·H₂O, wherein the pH is about 5.8. In some embodiments, the composition is sterile. In some embodiments, the composition is preservative-free.

In some embodiments of the aforementioned dosing methods or therapeutic methods, the subject has been diagnosed with or is at risk for developing dry age-related macular degeneration. In some embodiments, the subject has drusen. In some embodiments, subject has been diagnosed with or is at risk for developing geographic atrophy (GA). In some embodiments, the subject has been diagnosed with partial ellipsoid zone attenuation of one or more portions/zones of the macula of at least one eye. In some embodiments, the subject has been diagnosed with total ellipsoid zone attenuation of one or more portions/zone of the macula of at least one eye.

In some embodiments of the aforementioned dosing methods or therapeutic methods, the composition is administered to the subject once per day. In some embodiments of the aforementioned dosing methods or therapeutic methods, the composition is administered to the subject twice per day. In some embodiments of the aforementioned dosing methods or therapeutic methods, the composition is administered to the subject three times per day.

In some embodiments of the aforementioned dosing methods or therapeutic methods, the ophthalmic topical composition is sterile. In some embodiments, the ophthalmic topical composition is preservative-free. In some embodiments, the ophthalmic topical composition is sterile and preservative-free.

In some embodiments of the aforementioned dosing methods or therapeutic methods, the subject is human.

### EXAMPLES

The present technology is further illustrated by the following examples, which should not be construed as limiting in any way.

### Example 1-Topical Bevemipretide (SBT-272) Eye Drops in Development for Dry AMD: Phase 1 Trial Design (See Example 2, Below) Informed by these 13-Week GLP Study Results

### INTRODUCTION

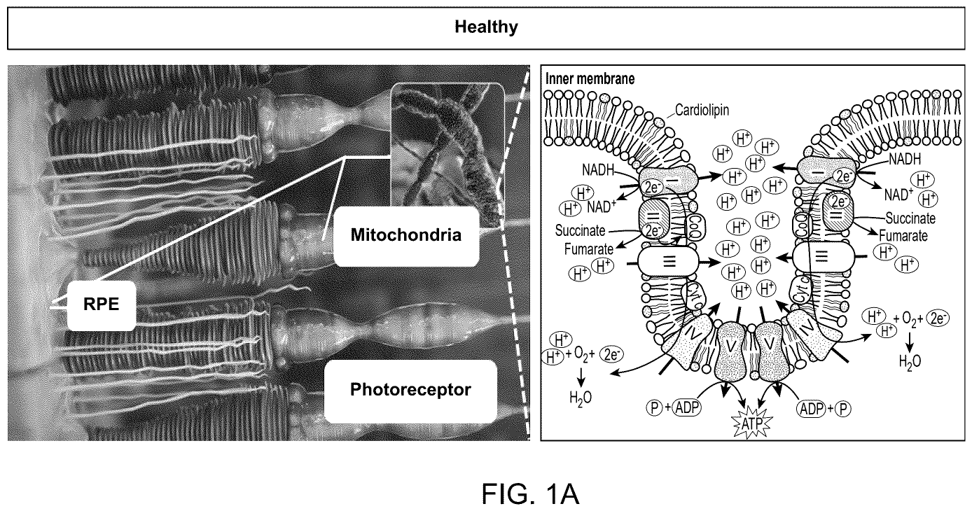

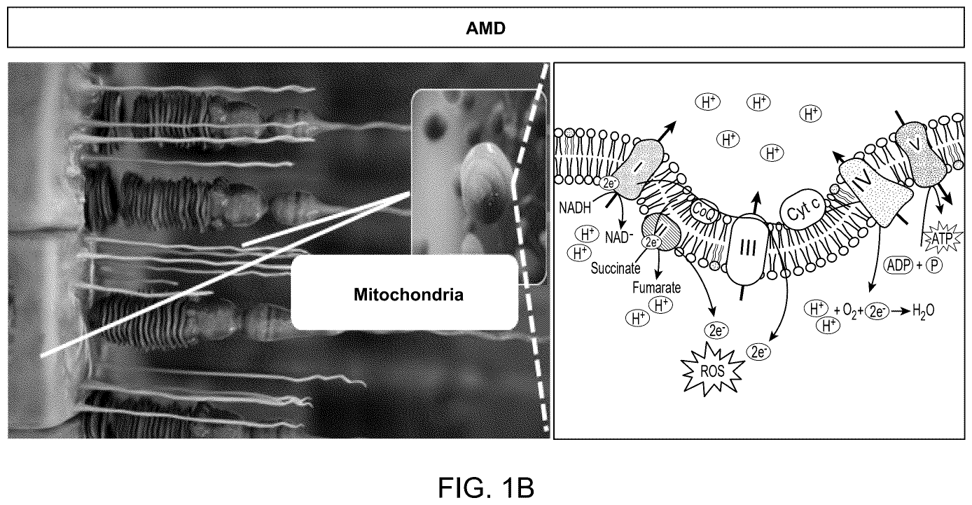

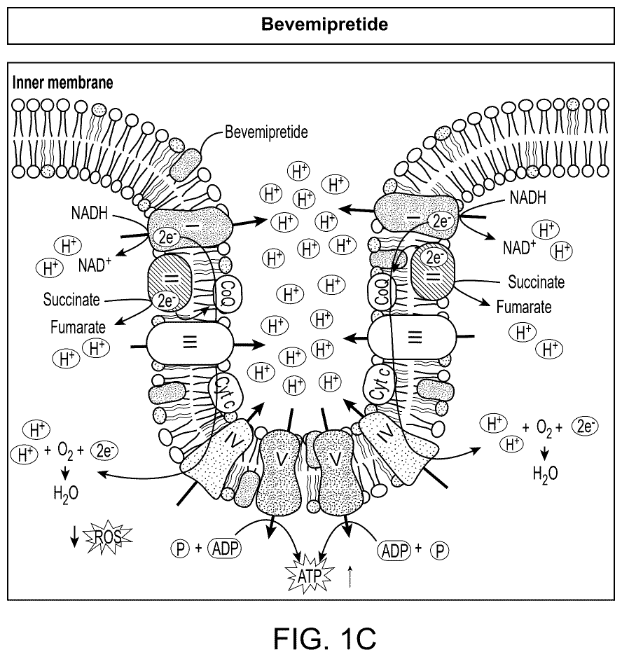

Mitochondrial dysfunction is an integral component of disease etiology in dry age-related macular degeneration (AMD). See, e.g., Kaarniranta, et al., 2020. Elamipretide, a mitochondria-targeted therapeutic, is being investigated in one global Phase 3 clinical trials for the treatment of dry AMD. See Ehlers, et al., 2024; and Mettu, et al., 2022. Bevemipretide (a.k.a., SBT-272), a next-generation mitochondria-targeting therapeutic, restores mitochondrial structure and function by associating with cardiolipin to reduce reactive oxygen species (ROS) and increase adenosine triphosphate (ATP) production (FIGS. 1A-1C). Previous studies demonstrated enhanced penetration to critical retinal tissues with bevemipretide through a topical eye drop formulation (data on file). See Ciallella, et al., 2024. This example evaluates the toxicology of bevemipretide for the treatment of dry AMD and outcomes following a recovery period.

### Methods:

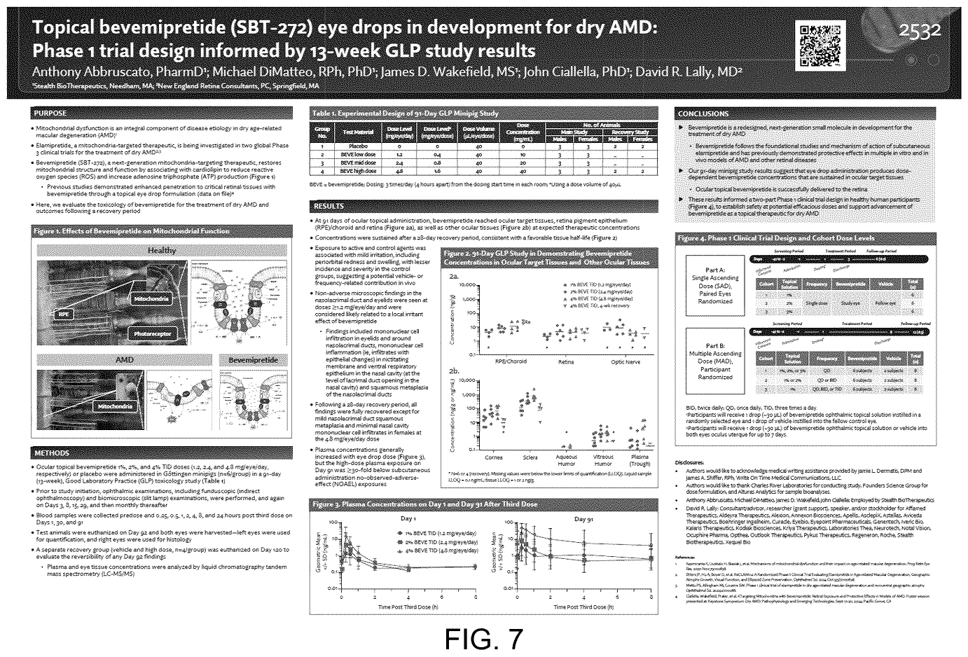

Bevemipretide ophthalmic topical solution 1%, 2%, and 4% TID doses (1.2, 2.4, and 4.8 mg/eye/day, respectively) or placebo were administered in Göttingen minipigs (n=6/group) in a 91-day (13-week), Good Laboratory Practice (GLP) toxicology study outlined in TABLE 1 (see also FIG. 7).

TABLE 1
Experimental Design of 91-Day GLP Minipig Study

| | | | Dose | Dose | | | | | |
|---|---|---|---|---|---|---|---|---|---|
| | | Dose Level | Level^{a} | Volume | Dose | No. of Animals | | | |
| Gp. | Test | (mg/eye/ | (mg/eye/ | (μL/eye/ | Concentration | Main Study | | Recovery Study | |
| No. | Material | day) | dose) | dose) | (mg/mL) | Males | Females | Males | Females |
| 1 | Placebo | 0 | 0 | 40 | 0 | 3 | 3 | 2 | 2 |
| 2 | BEVE | 1.2 | 0.4 | 40 | 10 | 3 | 3 | — | — |
| | low dose | | | | | | | | |
| 3 | BEVE | 2.4 | 0.8 | 40 | 20 | 3 | 3 | — | — |
| | mid dose | | | | | | | | |
| 4 | BEVE | 4.8 | 1.6 | 40 | 40 | 3 | 3 | 2 | 2 |
| | high dose | | | | | | | | |

Gp. Group; BEVE = bevemipretide; Dosing: 3 three times daily (4 hours apart) from the dosing start time in each room;
^{a}using a dose volume of 40 μL.

Prior to study initiation, ophthalmic examinations, including funduscopic (indirect ophthalmoscopy) and biomicroscopic (slit lamp) examinations, were performed on each subject, and again on Days 3, 8, 15, 29, and then monthly thereafter. Blood samples were collected predose and 0.25, 0.5, 1, 2, 4, 8, and 24 hours post third dose on Days 1, 30, and 91. Test animals were euthanized on Day 92 and both eyes were harvested-left eyes were used for quantification, and right eyes were used for histology. A separate 4-week recovery group (vehicle and high dose, n=4/group) was euthanized on Day 120 to evaluate the reversibility of any Day 92 findings. Plasma and eye tissue concentrations were analyzed by liquid chromatography tandem mass spectrometry (LC-MS/MS).

### Results:

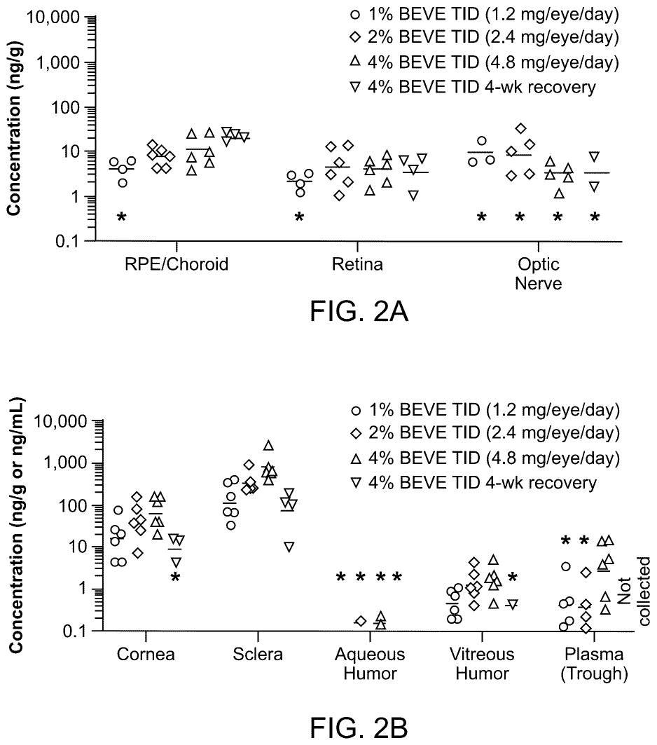

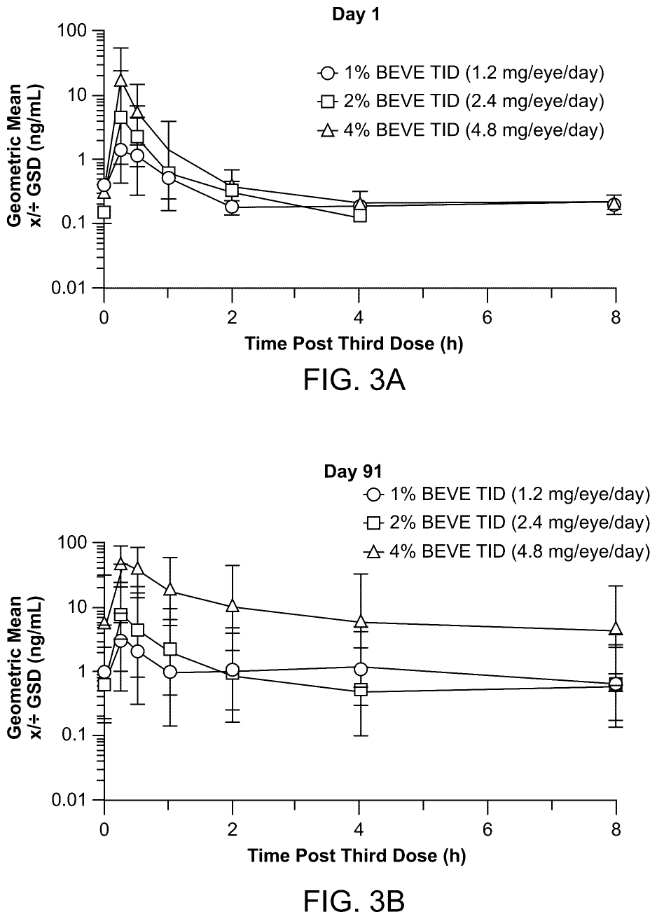

At 91 days of ocular topical administration, bevemipretide reached ocular target tissues, retina pigment epithelium (RPE)/choroid and retina (FIG. 2A), as well as other ocular tissues (FIG. 2B) at expected therapeutic concentrations. Concentrations were sustained after a 28-day (4-week) recovery period, consistent with a favorable tissue half-life (FIGS. 2A-2B). Exposure to active and control agents was associated with mild irritation, including periorbital redness and swelling, with lesser incidence and severity in the control groups, suggesting a potential vehicle- or frequency-related contribution in vivo. Non-adverse microscopic findings in the nasolacrimal duct and eyelids were seen at doses ≥1.2 mg/eye/day and were considered likely related to a local irritant effect of bevemipretide. Findings included mononuclear cell infiltration in eyelids and around nasolacrimal ducts, mononuclear cell inflammation (i.e., infiltrates with epithelial changes) in nictitating membrane and ventral respiratory epithelium in the nasal cavity (at the level of lacrimal duct opening in the nasal cavity) and squamous metaplasia of the nasolacrimal ducts. Following a 28-day recovery period, all findings were fully recovered except for mild nasolacrimal duct squamous metaplasia and minimal nasal cavity mononuclear cell infiltrates in females at the 4.8 mg/eye/day dose. Plasma concentrations generally increased with eye drop dose (FIGS. 3A-3B), but the high-dose plasma exposure on Day 91 was >30-fold below the no-observed adverse-effect-level (NOAEL) after subcutaneous administration in rat and monkey.

### Conclusions:

As demonstrated herein, the 91-day minipig study results demonstrate that eye drop administration produces dose-dependent bevemipretide concentrations that are sustained in ocular target tissues. Bevemipretide ophthalmic topical solution is successfully delivered to the retina; the eye tissue known to be most impacted by the deleterious effects of dry AMD. These results informed a two-part Phase 1 clinical trial design in healthy human participants as described in Example 2, to establish safety at potentially efficacious doses and support advancement of bevemipretide as a topical therapeutic for dry AMD.

Accordingly, these results demonstrate the efficacy of a method of dosing bevemipretide via eye drops in a subject in need thereof and delivery of the bevemipretide to the ocular tissues of the subject in quantities believed to be sufficient to produce a therapeutic benefit.

### Example 2-Two-Part, Phase 1, Randomized, Double-Masked, Vehicle-Controlled, Single and Multiple Ascending Dose Escalation Study to Evaluate the Safety, Tolerability, and Pharmacokinetics of Bevemipretide Topical Ophthalmic Solution in Healthy Subjects

Note: At the time of writing of this Example, the study described was not yet completed. With respect to the SAD portion of the study, all subjects had been dosed and treatment completed but not all results were finalized. With respect to the MAD portion, dosing of subjects had been completed for Cohorts 1 & 2 (also referred to herein as Cohorts B1 & B2, respectively). Cohort 3 (a.k.a. Cohort B3) of the MAD study was in progress, with only partial preliminary data available. Again, interim data was available and is reported in summary form but the study remained ongoing. Data and results presented in this Example 2 are based on information and analysis available at the time of writing. All data remained blinded so it was not possible to determine which eyes of subjects (SAD portion) or which subjects (MAD portion) received bevemipretide containing eye drops (i.e., active eye drops) and which received eye drops without any bevemipretide (i.e., placebo/control eye drops).

### I. Description of the Drug Product

The bevemipretide ophthalmic topical solution (i.e., the “drug product solution”) was supplied as pre-filled, single-use, blow-fill-seal (BFS) vials (the “drug product”). The drug product was sterile and preservative-free. The drug product was comprised of bevemipretide ophthalmic topical solution in an ophthalmic eye-drop dispenser (i.e., the drug product). The drug product was manufactured as a blow-fill-seal (BFS) vial/container closure system (i.e., a combination product under United States Food and Drug Administration (FDA) guidelines) under GMP manufacturing conditions in compliance with investigational new drug (IND) application filed with, and approved by, the FDA. The drug product was used in a phase I, human clinical trial (as described below) conducted in accordance with a study protocol reviewed and approved by the FDA.

Bevemipretide ophthalmic topical solution was a buffered, sterile solution with a target of pH 5.8 supplied in a single-use blow-fill-seal (BFS) vial. The drug product solution contained bevemipretide HCl drug substance (i.e., bevemipretide tris-HCl—the “drug substance”), sodium chloride (NaCl), sodium phosphate (NaH₂PO₄, in monohydrate form as used in preparation of the drug product solution, i.e., NaH₂PO₄·H₂O), and water for injection. For pH adjustment, either hydrochloric acid or sodium hydroxide were used, only as needed. The drug product solution was manufactured as a 1%, 2%, and 3% (i.e., 10 mg/mL, 20 mg/mL, and 30 mg/mL, respectively) strength of drug substance (based on the molecular weight of bevemipretide as a free base; i.e., salt and other impurities in the drug substance were compensated for in calculating the amount added to the production batch as described herein so that 10 mg/mL, 20 mg/mL, and 30 mg/mL of bevemipretide free-base was used in each formulation as indicated in the Tables below). TABLE 2 provides a summary description of each drug product used in the clinical study described below, and TABLE 3 outlines the components and composition of the drug product solution in unit and batch format.

The container closure system was a blow-fill-seal (BFS) vial (BFS vial comprised of virgin Purell Low Density Polyethylene (LDPE) Resin) prepared using custom designed equipment at the manufacturer. Each single-use BFS vial contained nominally 0.32 mL of bevemipretide drug product solution (i.e., ophthalmic topical solution).

BFS technology is an advanced aseptic process that limits exposure of product to the environment and conditions that may affect product sterility through the use of manufacturing automation and design. In the BFS process, the vials can be formed, filled from a sterile solution batch and sealed in a continuous automated aseptic process that limits exposure of product to the environment. The BFS process consists of the following operations: parison formation from molten LDPE by extrusion, container formation using appropriate mold, product filling and container sealing. All these operations can be conducted in Class 100 (ISO5) environments using HEPA filtered air. As described, the drug product exits the manufacturing equipment in a sealed, sterile, ready for use form.

### II. Physiochemical Biological Properties of the Drug Product Solution

The bevemipretide ophthalmic topical solution (i.e., drug product solution) was a non-viscous, substantially clear, water-like solution formulated at less than the saturation concentration for bevemipretide; thus drug substance physical properties had no impact on the drug product performance. The pH target range was pH 5.7-5.9, to provide adequate solution stability over a period of at least 6 months. The osmolality of the formulated solution was 200-400 mOsm/kg and was consistent with ophthalmic topical administration.

TABLE 2
Summary Description of Bevemipretide Drug Products Used in the Clinical Study

| | Concentration of | | |
|---|---|---|---|
| | Bevemipretide | | Primary Container |
| Product Name | (free base) | Nominal Fill | Closure System |
| Bevemipretide | 1% (10 mg/mL) | 0.32 mL | Blow-fill-seal (BFS) ≈ 0.5 |
| ophthalmic topical | 2% (20 mg/mL) | | mL vial with twist-off |
| solution | 3% (30 mg/mL) | | closure comprised of |
| | | | virgin Purell Low Density |
| | | | Polyethylene (LDPE) |
| | | | 1840H Resin |

TABLE 3
Components and Composition of Bevemipretide Ophthalmic Topical Solution - Batch Formula

| | | 1% (10 mg/mL) | | | | 2% (20 mg/mL) | | | | 3% (30 mg/mL) | | | |
|---|---|---|---|---|---|---|---|---|---|---|---|---|---|
| | | formulation | | | | formulation | | | | formulation | | | |
| | | Unit | | Batch | | Unit | | Batch | | Unit | | Batch | |
| | | Formula | | Formula ² | | Formula | | Formula ² | | Formula | | Formula ² | |
| Component | Function | (mg/mL) | | (g) | | (mg/mL) | | (g) | | (mg/mL) | | (g) | |
| Bevemipretide ¹ | Drug | 10.0 | mg | 9.90 | g | 20.0 | mg | 19.80 | g | 30.0 | mg | 29.70 | g |
| | Substance | | | | | | | | | | | | |
| Sodium | Tonicity | 5.3 | mg | 5.25 | g | 3.4 | mg | 3.37 | g | 1.5 | mg | 1.46 | g |
| Chloride | adjustment | | | | | | | | | | | | |
| Sodium | pH Buffer | 4.16 | mg | 4.12 | g | 4.16 | mg | 4.12 | g | 4.16 | mg | 4.12 | g |
| Phosphate | | | | | | | | | | | | | |
| Monobasic | | | | | | | | | | | | | |
| Monohydrate | | | | | | | | | | | | | |
| Sodium | pH | Qs.³ to pH 5.8 | | | | | | | | | | | |
| Hydroxide | Adjustment | | | | | | | | | | | | |
| Hydrochloric | | | | | | | | | | | | | |
| Acid | | | | | | | | | | | | | |

| | | | | | | | |
|---|---|---|---|---|---|---|---|
| Water for | Solvent | Qs.³ to | Qs.³ to | Qs.³ to | Qs.³ to | Qs.³ to | Qs.³ to |
| Injection | | 1 mL | 1.0 kg ⁴ | 1 mL | 1.0 kg ⁴ | 1 mL | 1.0 kg ⁴ |

¹ The amount of bevemipretide as a free base, from bevemipretide tris hydrochloride salt, was used and adjusted for potency (based upon bevemipretide free-base in the drug substance (i.e., API) as determined by the applicable Certificate of Analysis) using the formula: Target batch volume (L) × target concentration (mg/mL) × (1 ÷ API potency as free base (decimal)) = Amt API to be added (g)
² Batch is prepared gravimetrically; quantities are for approximately 0.991 L for a 1.0 kg batch.
³Qs. = Add as needed
⁴ Approximately 0.991 L of bevemipretide bulk drug product solution (i.e., ophthalmic topical solution). Bevemipretide ophthalmic topical solution has a density of 1.01 g/mL.

### III. Manufacturing Process Description of the Drug Product

a. Active-Bevemipretide Ophthalmic Topical Solution

Bevemipretide ophthalmic topical solution was manufactured using the manufacturing process provided schematically in FIG. 4. Specifics of the relative amounts used to produce 1 kilogram of bevemipretide ophthalmic topical solution are provided in TABLE 3, but this is generic and was scaled based on the batch size needed to produce sufficient quantities of single-use blow-fill-seal vials for the entire study. The manufacturing process included the elements of raw materials release and dispensing, compounding, filtration, and a BFS filling process with in-process controls (IPCs).

The drug product was manufactured preservative-free. To ensure sterility, the bevemipretide ophthalmic topical solution was filtered through two filters (in serial) and into the BFS apparatus to produce the BFS vials containing the sterile, preservative-free bevemipretide ophthalmic topical solution. In the BFS process, the single-patient-use vials were formed, filled and sealed in a continuous automated aseptic process that limits exposure of drug product to the environment. In the BFS apparatus used to produce the drug product, a numerically coded five-vial card (five vials attached together) was created as the output. Numerical coding was used to distinguish each batch from the other batches (e.g., distinguish between the 1% batches, 2% batches, 3% batches, and 0% batches (0% being the placebo/vehicle control doses). Those five-vial cards were then packaged in a foil pouch with protective barrier properties for storage, shipping and distribution. A description of the primary packaging is provided in TABLE 4. An illustration of various views of the individual BFS vial and the five-vial card produced using the BFS process containing the bevemipretide ophthalmic topical solution is presented in FIG. 5.

Each BFS vial used in this study was a sterile, preservative-free single-use container comprising enough bevemipretide ophthalmic topical solution for administration according to the single ascending dose (SAD) or multiple ascending dose (MAD) protocol discussed below (whether delivered to a single eye or both eyes during a dose administration). Access to the bevemipretide ophthalmic topical solution for administration was attained by removing one vial from the other vials of the 5-vial card, and twisting off the top of the individual vial to expose the bevemipretide ophthalmic topical solution for administration as an eye drop to the subject's eye. A preservative-free formulation was selected for the study to avoid potential adverse events that a preservative might cause and other possible complications that might arise with preservative use in the study of the target population. A single-use vial was selected to maintain sterility of the bevemipretide ophthalmic topical solution until the time of administration given the absence of a preservative. Stability testing was performed to ensure sterility and compliance with other specifications of the drug product during the entire term of the clinical study. The BFS manufacturing process was selected to accomplish these objectives for the drug product.

TABLE 4
Description of the Bevemipretide Ophthalmic
Topical Solution Primary Packaging

| Component | Description |
|---|---|
| Vial | Virgin Purell Low Density Polyethylene (LDPE) |
| | 1840H Resin |
| Vial/Closure | Blow fill seal (BFS) ≈ 0.5 mL vial with twist-off |
| | closure |
| Foil Pouch | 7.5″ blank Flexi 2114 polyester/aluminum |
| | foil/polyethylene with protective properties |

### Vial, 0.5 mL (Resin)

Virgin Purell Low Density Polyethylene (LDPE) 1840H Resin was used to form the BFS vial/twist-off top container (FIG. 5). The resin met the requirements of USP <661> (for physicochemical properties) and USP <87> and <88> (biological reactivity) and thus was suitable for its intended use. The resin also meets the criteria in 21 CFR 176 and 177.1520 for food contact applications and is widely used in approved ophthalmic topical solutions.

Phthalates were not used in the manufacture or formulation of the resin.

### Vial and Vial-Card

Clinical trial subjects received a sufficient number of the five-vial card units (FIG. 5) and instructions necessary to complete the phase of the clinical trial for which they participated under proper supervision as dictated by the clinical study protocol. Briefly, each BFS-vial contained a sterile, preservative-free single-use dose of bevemipretide ophthalmic topical solution (or placebo/vehicle) for administration as an eye drop but subjects and the clinicians were blinded to whether the active or placebo batch was used for dosing. In the form used, the bevemipretide ophthalmic topical solution (or placebo control) could be self-administered or administered by a third party in accordance with the study protocol. The subject (or clinical staff responsible for administration/dosing) was instructed to remove one of the single-use BFS-vials from the five-vial card, remove the cap and administer the dose (to one or both eyes as appropriate). After administration of the dose from the single-use BFS-vial in accordance with the study protocol, the study participant was instructed to dispose of any remaining material and open a new single-use BFS-vial when the next dose was required (not applicable in the SAD study).

### IV. Description of the Placebo/Vehicle Drug Product & Placebo/Vehicle Drug Product Solution

Placebo/vehicle drug product was supplied as pre-filled, single-use, blow-fill-seal (BFS) vials (the “placebo drug product”). The placebo drug product was sterile and preservative-free. The placebo drug product was comprised of placebo ophthalmic topical solution in an ophthalmic eye-drop dispenser (i.e., the placebo drug product). The placebo drug product was manufactured as a blow-fill-seal (BFS) vial/container closure system (i.e., a combination product under United States Food and Drug Administration (FDA) guidelines) under GMP manufacturing conditions in compliance with investigational new drug (IND) application filed with, and approved by, the FDA. The placebo drug product was used in a phase I, human clinical trial (as described below) conducted in accordance with a study protocol reviewed and approved by the FDA. The placebo drug product was identical to (and therefore indistinguishable from) the “active” drug product in all physical attributes, except it contained no bevemipretide, as discussed below.

Placebo (i.e., vehicle control) ophthalmic topical solution was a buffered, sterile solution with a target of pH 5.8 supplied in a single-use blow-fill-seal (BFS) vial. The placebo drug product solution contained sodium chloride (NaCl), sodium phosphate (NaH₂PO₄, in monohydrate form as used in preparation of the placebo drug product solution, i.e., NaH₂PO₄·H₂O), and water for injection. For pH adjustment, either hydrochloric acid or sodium hydroxide were used, only as needed. Because no bevemipretide was contained in the placebo drug product or placebo drug product solution, only one placebo/vehicle was needed regardless of which active (i.e., 1%, 2%, or 3% bevemipretide ophthalmic topical solution) was being tested against this placebo (i.e., vehicle control) ophthalmic topical solution in the study. TABLE 5 outlines the components of the placebo drug product solution and its/their function and provides information for batch formulation in unit and batch format (unit formulation is volumetric and batch formulation is gravimetrically-based.

TABLE 5

| | | Unit | | Batch | |
|---|---|---|---|---|---|
| | | Formula | | Formula ¹ | |
| Component | Function | (mg/mL) | | (g) | |
| Sodium Chloride | Tonicity | 7.2 | mg | 7.128 | g |
| | adjustment | | | | |
| Sodium Phosphate | pH Buffer | 4.16 | mg | 4.118 | g |

| Monobasic | | | |
|---|---|---|---|
| Monohydrate | | | |
| Sodium Hydroxide | pH Adjustment | Qs. to pH 5.8 | |
| Hydrochloric Acid | | | |
| Water for Injection | Solvent | Qs. to | Qs. to |
| | | 1 mL | 1.0 kg ² |

¹ Batch is prepared gravimetrically; quantities are for approximately 0.991 L (1.0 kg) batch
² Approximately 0.991 L of placebo drug product solution at a density of 1.01 g/mL

The container closure system was a blow-fill-seal (BFS) vial (BFS vial comprised of virgin Purell Low Density Polyethylene (LDPE) Resin) prepared using custom designed equipment at the manufacturer previously discussed with respect to manufacture of the “active” drug product (i.e., BFS vials containing the 1%, 2%, or 3% bevemipretide ophthalmic topical solution), described previously. The placebo drug product was manufactured in the same manner as was the 1%, 2%, or 3% bevemipretide drug product except that the BFS equipment was fed the placebo drug product solution instead of an active drug containing solution. Hence, the placebo drug product was indistinguishable from the 1%, 2%, or 3% bevemipretide drug product except in how the BFS vials were numerically coded. In a similar vein, the 1%, 2%, and 3% bevemipretide drug products were indistinguishable from each other and from the placebo drug product, except in how they were numerically coded during manufacture.

### V. Manufacturing Controls

All manufacturing was performed in strict compliance with the approved protocols under applicable Good Manufacturing Practices (GMP). All active and vehicle control drug products were used in the clinical study discussed below prior to their approved expiry dates as determined by stability testing of prior batches and continued stability testing of the batches used in the study.

### VI. Study Protocol:

### 1. Summary

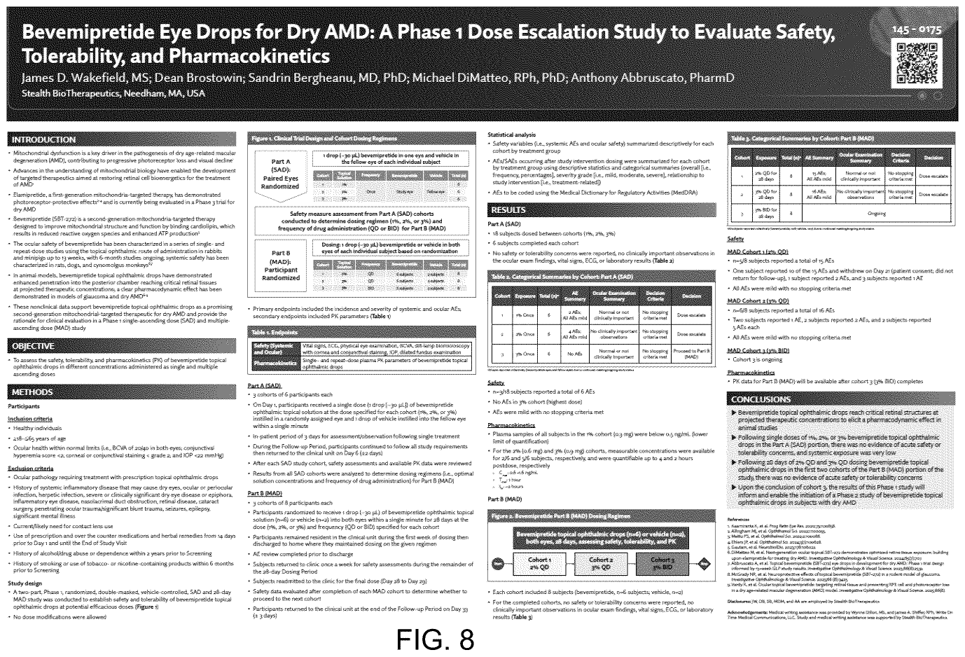

This was a two-part, Phase 1, randomized, double-masked, vehicle-controlled, single and multiple ascending dose escalation study to evaluate the safety, tolerability, and pharmacokinetics of bevemipretide ophthalmic topical solution in healthy subjects (see FIG. 8). The study was designed to examine bevemipretide eye drops in different concentrations administered as single and multiple doses. This study was intended to help determine the appropriate highest safe and tolerable concentration of bevemipretide ophthalmic topical solution suitable for use in the subsequent human clinical studies in the target patient population. The objectives and endpoints of the study are listed in TABLE 6.

TABLE 6
Study Objectives and Endpoints

| Objectives | Endpoints |
|---|---|
| Primary | |
| To evaluate the safety and tolerability of | The incidence and severity of systemic and |
| single and multiple ascending doses of | ocular AEs: |
| bevemipretide ophthalmic topical solution | All safety endpoints (including AEs, |
| in healthy subjects | vital signs, ECG, safety laboratory) |
| | Ocular safety assessments (physical |
| | examination of eyes, BCVA, slit lamp |
| | biomicroscopy with corneal and |
| | conjunctival staining, IOP, dilated |
| | fundus examination) |
| Secondary | |
| To evaluate the plasma PK of single and | PK parameters: |
| multiple ascending doses of bevemipretide | Single- and repeat -dose plasma PK |
| ophthalmic topical solution in healthy | parameters, including but not limited to: |
| subjects | Cₘₐₓ, Tₘₐₓ, AUC₂₄, AUCₗₐₛₜ, |
| | AUC_{inf}, AUCₜₐᵤ, Cₗₐₛₜ, Tₗₐₛₜ, t½ |

Table Legend: AE: adverse event; AUC24: area under the plasma concentration-time curve from time zero to 24 hours postdose; AUCinf: area under the plasma concentration curve from time zero to infinity; AUClast: area under the plasma concentration-time curve from time zero to time of last quantifiable plasma concentration; AUCtau: area under the plasma concentration-time curve within a dosing interval; BCVA: best-corrected visual acuity; Clast: last observed quantifiable concentration in plasma; Cmax: maximum observed concentration in plasma; ECG: electrocardiogram; IOP: intraocular pressure; PK: pharmacokinetic(s); Tlast: time of last quantifiable concentration in plasma; Tmax: time of maxium concentration in plasma, t½: apparent terminal half-life.

### 2. Summary Overall Study Design:

Part A consisted of 3 single ascending dose (SAD) cohorts of 6 subjects each. Each participant received 1 drop (about 30 μL) of bevemipretide ophthalmic topical solution instilled in a randomly selected eye and 1 drop of vehicle instilled into the fellow control eye within a single minute.

Part B consisted of 3 multiple ascending dose (MAD) cohorts of 8 subjects each, randomized in a 3:1 ratio of bevemipretide ophthalmic topical solution to vehicle (no results were available for the 3^{rd} cohort of 8 subjects at the time of filing of this application). In this part B, one drop of bevemipretide ophthalmic topical solution or vehicle was administered to both eyes (oculus uterque [OU]) of the subject within a single minute for up to 28 days. Study intervention was administered once-daily (QD), or twice-daily (BID), at frequencies and dose concentrations based on safety and tolerability results from previous cohorts. The specifics of each cohort are described below in TABLES 8 & 9.

### 3. Number of Subjects Study Cohorts and Duration:

Part A consisted of 3 SAD cohorts of 6 subjects each (18 total subjects); the study duration was up to 48 days, including a treatment period per cohort of 3 days.

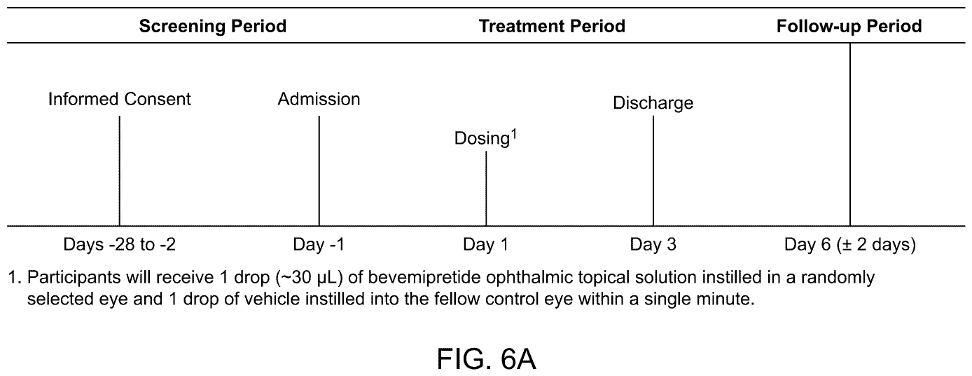

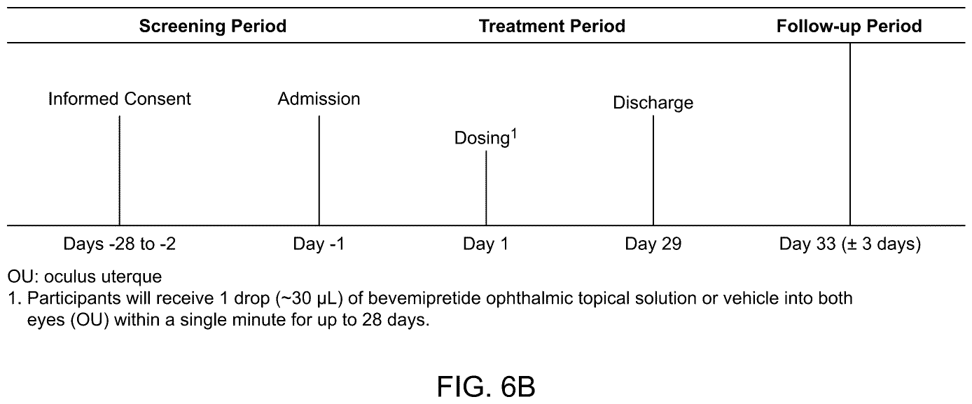

With reference to FIG. 6A, a schematic summary of the Part A-Single Ascending Dose (SAD) portion of the study is provided. Each of the three SAD cohorts followed this timeline.

Part B consisted of 3 MAD cohorts of 8 subjects each (24 total subjects); the study duration was up to 117 days, including a treatment period per cohort of 29 days.

With reference to FIG. 6B, a schematic summary of the Part B-Multiple Ascending Dose (MAD) portion of the study. Each of the three MAD cohorts followed/were following this timeline.

### 4. Inclusion/Exclusion Criteria Applied To Determine Subject Eligibility:

Inclusion criteria
Healthy individuals
≥18-≤65 years of age
Ocular health within normal limits (i.e., BCVA of 20/40 in both eyes; conjunctival hyperemia score <2; corneal or conjunctival staining <grade 2; and IOP <22 mmHg)
   Exclusion criteria
Ocular pathology requiring treatment with prescription ophthalmic topical drops
History of systemic inflammatory disease that may cause dry eyes, ocular or periocular infection, herpetic infection, severe or clinically significant dry eye disease or epiphora, inflammatory eye disease, nasolacrimal duct obstruction, retinal disease, cataract surgery, penetrating ocular trauma/significant blunt trauma, seizures, epilepsy, significant mental illness
Current/likely need for contact lens use
Use of prescription and over the counter medications and herbal remedies from 14 days prior to Day 1 and until the End of Study Visit
History of alcohol/drug abuse or dependence within 2 years prior to Screening
History of smoking or use of tobacco- or nicotine-containing products within 6 months prior to Screening

### 5. Study Design:

This was a phase 1, single-center, randomized, double-masked, vehicle-controlled, single and multiple ascending dose escalation study to evaluate the safety, tolerability, and pharmacokinetics of topically administered bevemipretide ophthalmic topical solution in healthy subjects.

Part A consisted of 3 single ascending dose (SAD) cohorts of 6 subjects each. Each subject received 1 drop (~30 μL) of bevemipretide ophthalmic topical solution administered in a randomly selected eye and 1 drop of vehicle administered into the fellow control eye within a single minute. Each subject was administered a single dose for a single day. Cohort 1 was administered the 1% bevemipretide ophthalmic topical solution to one randomized eye and vehicle to the other eye. Cohort 2 was administered the 2% bevemipretide ophthalmic topical solution to one randomized eye and vehicle to the other eye. Cohort 3 was administered the 3% bevemipretide ophthalmic topical solution to one randomized eye and vehicle to the other eye.

Part B consisted of 3 multiple ascending dose (MAD) cohorts of 8 subjects each, randomized in a 3:1 ratio of bevemipretide ophthalmic topical solution to vehicle (i.e., 6 randomized subjects received only bevemipretide ophthalmic topical solution over the entire 28-day treatment period and 2 randomized subjects received only vehicle over the entire 28-day treatment period). One drop (~30 μL) of bevemipretide ophthalmic topical solution or vehicle was administered into both eyes of the subject at each dosing event within a single minute for up to 28 days. Study intervention was administered once-daily (QD), or twice-daily (BID). Cohort 1 was administered 2% bevemipretide ophthalmic topical solution or vehicle once-daily (QD) for each of the 28 days of treatment. Cohort 2 was administered 3% bevemipretide ophthalmic topical solution or vehicle once-daily (QD) for each of the 28 days of treatment. Cohort 3 is being administered 3% bevemipretide ophthalmic topical solution or vehicle twice-daily (BID) for each of the 28 days of treatment as part of the still ongoing investigational cohort of the study.

In each study part there were 3 periods:
Screening Period
Treatment Period
   0 Part A-SAD, paired eyes randomized
   0 Part B-28-day MAD, Subjects randomized
Follow-up Period
   a. Screening Period

The study required obtaining written informed consent from all subjects prior to the Screening Visit. Screening procedures could be completed on more than one day, as long as all procedures are completed during the Screening Period. All other visits were completed over a 1- or 2-day period, at the discretion of the principal investigator, as long as all procedures were completed during the allowable window for that visit.

The screening examination was performed no more than 28 days prior to baseline. For applicable female subjects, urine pregnancy testing was performed prior to initiation of treatment. All subjects submitted a urine sample for screening of alcohol and/or substances of abuse. The day of randomization was defined as Day 1 in each part of the study. Additional data was collected from a complete pre-treatment examination, consisting of vital signs, physical examination, ocular examinations, serum pregnancy test for women of child-bearing potential, routine blood chemistries and urinalysis. Subjects were considered enrolled in the study upon randomization.

b. Treatment Period

### Part A-Single Ascending Dose, Paired Eyes Randomized

Subjects who meet the eligibility criteria evaluated during the Screening Period were selected to enter the Treatment Period beginning on Day 1, and paired eyes were randomized to receive either bevemipretide (study eye) or vehicle (fellow eye) at the dose level specified for that cohort as shown in TABLE 7.

Subjects remained resident in the clinical unit until completion of the 48-hour postdose assessments on Day 3. An adverse event (AE) review was completed prior to discharge.

TABLE 7
Study Intervention Administration for Part A -
Single Ascending Dose, Paired Eyes Randomized

| | | | | | | Total |
|---|---|---|---|---|---|---|
| | | | | | | Number |
| | Topical | Total | | | | of |
| Cohort | Solution | Dose¹ | Frequency | Bevemipretide | Vehicle | Subjects |
| A1 | 1% | ~0.30 mg | Single | Study eye | Fellow | 6 |
| | (10 mg/mL) | | dose | | eye | |
| A2 | 2% | ~0.60 mg | Single | Study eye | Fellow | 6 |
| | (20 mg/mL) | | dose | | eye | |
| A3 | 3% | ~0.90 mg | Single | Study eye | Fellow | 6 |
| | (30 mg/mL) | | dose | | eye | |

¹Subjects received 1 drop (~30 μL) of bevemipretide ophthalmic topical solution instilled in a randomly selected eye and 1 drop of vehicle instilled into the fellow control eye within a single minute.

Sentinel dosing was performed, such that the first 2 subjects in each cohort of Part A (Cohorts A1, A2, and A3) received bevemipretide ophthalmic topical solution or vehicle randomized to study eye or fellow eye, and safety data (a minimum of AEs, vital signs, best-corrected visual acuity (BCVA), and IOP) collected through 24 hours after dosing. This data was reviewed by the principal investigator or designee prior to proceeding to dosing the remaining subjects in the cohort.

After each cohort was studied and before any subsequent cohort was dosed, the available masked safety data up to 48 hours postdose from all subjects in the preceding cohort was reviewed.

All three cohorts of the SAD study were dosed as expected as there were no safety concerns that arose. Detailed (yet preliminary) results are discussed in the Results section below.

### Part B-Multiple Ascending Dose, Participant Randomized

Subjects who meet the eligibility criteria evaluated during the Screening Period were selected to enter the Treatment Period beginning on Day 1 and were randomized to receive either bevemipretide or vehicle in both eyes at the dose level and frequency specified for that cohort (TABLE 8).

Subjects remained resident in the clinical unit until completion of the 24-hour postdose assessments and self-administration of the first dose on Day 8. Treatment continued by self-administration at home and subjects returned once a week for in-clinic administration and safety assessments during the remainder of the 4-week dosing period. Subjects were readmitted to the clinic for the final dose (Day 28 to Day 29). An AE review was completed prior to discharge. Safety data was evaluated after completion of each MAD cohort to determine whether to proceed to the next cohort. Participants returned to the clinical unit at the end of the Follow-up Period on Day 33 (+3 days).

TABLE 8
Study Intervention Administration for Part B -
Multiple Ascending Dose, Participant Randomized

| | | | | Number of Subjects | | |
|---|---|---|---|---|---|---|
| Cohort | Topical Solution | Total Dose¹ | Frequency | Bevemipretide | Vehicle | Total |
| B1 | 2% | ~0.60 | QD | 6 | 2 | 8 |
| | (20 mg/mL) | mg/eye/day | | | | |
| B2 | 3% | ~0.90 | QD | 6 | 2 | 8 |
| | (30 mg/mL) | mg/eye/day | | | | |
| B3 | 3% | ~1.80 | BID | 6 | 2 | 8 |
| | (30 mg/mL) | mg/eye/day | | | | |

BID: twice-daily; QD: once-daily
¹Subjects received 1 drop (~30 μL) of bevemipretide ophthalmic topical solution or vehicle into both eyes (OU) within a single minute for up to 28 days.

The scientific review board (SRB) performed a joint dose escalation review (masked data review) during the study. Safety, tolerability (e.g., ophthalmic safety parameters, AEs, laboratory results, ECG) and PK data available up to Day 6 for at least 15 out of 18 Subjects in all cohorts in Part A (SAD) was examined to support the dose escalation decisions for initiation of Cohort B1 and subsequent cohorts.

At the end of each cohort for Part B (MAD), all predefined safety data available up to Day 33 for all Subjects was examined in a masked fashion for a decision on continuing dose evaluation in the study. Safety data from at least 6 out of 8 Subjects was evaluated for a decision on continuing to the next cohort of Part B (MAD). Planned dose levels, dosing frequencies, and number of drops per eye were determined based on the emerging safety data. All three cohorts proceeded as described in TABLE 8 as no significant AEs were recorded in the first two cohorts. Detailed (yet preliminary) results are discussed in the Results section below.

c. Follow-up Period

The Follow-up Period began after the end-of-treatment (EOT) discharge from the clinical unit. During the Follow-up Period, subjects continued to follow all study requirements. All subjects returned to the clinical unit on Day 6 in Part A (SAD, +2 days) and Day 33 in Part B (MAD, +3 days).

### 6. Statistical Analysis:

Safety variables (i.e., systemic AEs and ocular safety) summarized descriptively for each cohort by treatment group.
AEs/SAEs occurring after study intervention dosing were summarized for each cohort by treatment group using descriptive statistics and categorical summaries (overall [i.e., frequency, percentages], severity grade [i.e., mild, moderate, severe], relationship to study intervention [i.e., treatment-related])
AEs to be coded using the Medical Dictionary for Regulatory Activities (MedDRA)

### 7. Preliminary Results (Reminder; all Samples Remain Masked):

### Part A (SAD)·

18 subjects dosed between cohorts (1%, 2%, 3%)
6 subjects completed each cohort
No safety or tolerability concerns were reported, no clinically important observations in the ocular exam findings, vital signs, ECG, or laboratory results (TABLE 9).

TABLE 9
Categorical Summaries by Cohort: Part A (SAD)

| | | | | Ocular | | |
|---|---|---|---|---|---|---|
| | | | AE | Examination | Decision | |
| Cohort | Exposure | Total (n)^{a} | Summary | Summary | Criteria | Decision |
| 1 | 1% Once | 6 | 2 AEs; | Normal or | No | Dose |
| | | | All AEs | not clinically | stopping | escalate |
| | | | mild | important | criteria | |
| | | | | | met | |
| 2 | 2% Once | 6 | 4 AEs; | No clinically | No | Dose |
| | | | All AEs | important | stopping | escalate |
| | | | mild | | criteria | |
| | | | | | met | |
| 3 | 3% Once | 6 | No AEs | Normal or | No | Proceed to |
| | | | | not clinically | stopping | Part B |
| | | | | important | criteria | (MAD) |
| | | | | | met | |

^{a}All eyes reported collectively (bevemipretide eyes and fellow eyes) due to continued masking/ongoing study status.

### Safety

n= 3/18 subjects reported a total of 6 AEs
No AEs in 3% cohort (highest dose)
AEs were mild with no stopping criteria met

### Pharmacokinetics

Plasma samples of all subjects in the 1% cohort (0.3 mg) were below 0.5 ng/ml (lower limit of quantification)
For the 2% (0.6 mg) and 3% (0.9 mg) cohorts, measurable concentrations were available for 2/6 and ⅚ subjects, respectively, and were quantifiable up to 4 and 2 hours postdose, respectively.
   Cₘₐₓ: 0.6-1.6 ng/ml
   Tₘₐₓ: 1 hour
   t_{1/2}: ~2 hours

Part B (MAD)· Each cohort included 8 subjects (bevemipretide, n=6 subjects; vehicle, n=2)· For the completed cohorts, no safety or tolerability concerns were reported, and no clinically important observations in ocular exam findings, vital signs, ECG, or laboratory results (TABLE 10).

TABLE 10
Categorical Summaries by Cohort: Part B (MAD)

| | | | | Ocular | | |
|---|---|---|---|---|---|---|
| | | | AE | Examination | Decision | |
| Cohort | Exposure | Total (n)^{a} | Summary | Summary | Criteria | Decision |
| 1 | 2% QD | 8 | 15 AEs | Normal or | No | Dose |
| | for 28 | | All AEs | not clinically | stopping | escalate |
| | days | | mild | important | criteria | |
| | | | | | met | |
| 2 | 3% QD | 8 | 16 AEs; | No clinically | No | Dose |
| | for 28 | | All AEs | important | stopping | escalate |
| | days | | mild | observations | criteria | |
| | | | | | met | |
| 3 | 3% BID | 8 | Ongoing | | | |
| | for 28 | | | | | |
| | days | | | | | |

^{a}All subjects reported collectively (bevemipretide, n = 6; vehicle, n = 2) due to continued masking/ongoing study status.

Safety

### MAD Cohort 1 (2% Once Per Day (QD))

n=⅝ subjects reported a total of 15 AEs
One subject reported 10 of the 15 AEs and withdrew on Day 21 (patient consent; did not return for follow-up), 1 subject reported 2 AEs, and 3 subjects reported 1 AE
All AEs were mild with no stopping criteria met
MAD Cohort 2 (3% QD) n= 6/8 subjects reported a total of 16 AEs
Two subjects reported 1 AE, 2 subjects reported 2 AEs, and 2 subjects reported 5 AEs each
All AEs were mild with no stopping criteria met

### MAD Cohort 3 (3% BID)

Cohort 3 is ongoing

### Pharmacokinetics

PK data for Part B (MAD) will be available after Cohort 3 (3% BID) completes

### 8. Conclusions:

Bevemipretide ophthalmic topical drops reach critical retinal structures at projected therapeutic concentrations to elicit a pharmacodynamic effect, in animal studies.
Following single doses of 1%, 2%, or 3% bevemipretide ophthalmic topical drops in the Part A SAD portion, there was no evidence of acute safety or tolerability concerns, and systemic exposure was very low.
Following 28 days of 2% QD and 3% QD dosing bevemipretide ophthalmic topical drops in the first two cohorts of the Part B MAD portion of the study, there was no evidence of acute safety or tolerability concerns.
Upon the conclusion of cohort 3, the results of this Phase 1 study will inform and enable the initiation of a Phase 2 study of bevemipretide ophthalmic topical drops in subjects with dry AMD.

FINAL NOTE, at the time of writing this Example, Cohort 3 (a.k.a., Cohort B3) of the MAD study was in progress and assessing 3% bevemipretide at BID frequency for safety and tolerability.

### EQUIVALENTS

The present technology is not to be limited in terms of the particular embodiments described in this application, which are intended as single illustrations of individual aspects of the present technology. Many modifications and variations of this technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that the present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

All patents, patent applications, provisional applications, and publications referred to or cited herein are incorporated by reference in their entirety, including all figures and tables, to the extent they are not inconsistent with the explicit teachings of this specification. Other embodiments are set forth within the following claims.

### REFERENCES

1. Kaarniranta K, Uusitalo H, Blasiak J, et al. Mechanisms of mitochondrial dysfunction and their impact on age-related macular degeneration. Prog Retin Eye Res. 2020 November;79:100858
2. Ehlers JP, Hu A, Boyer D, et al. ReCLAIM-2: A Randomized Phase II Clinical Trial Evaluating Elamipretide in Age-related Macular Degeneration, Geographic Atrophy Growth, Visual Function, and Ellipsoid Zone Preservation. Ophthalmol Sci. 2024 Oct. 9;5 (1): 100628
3 Mettu PS, Allingham MJ, Cousins SW. Phase 1 clinical trial of elamipretide in dry age-related macular degeneration and noncentral geographic atrophy. Opththalmol Sci. 2022; 2:100086
4. Ciallella, Wakefield, Prater, et al. Targeting Mitochondria with Bevemipretide: Retinal Exposure and Protective Effects in Models of AMD. Poster session presented at: Keystone Symposium: Dry AMD: Pathophysiology and Emerging Technologies. Sep. 17-20, 2024; Pacific Grove, CA 5 WO 2023/069549 & US 2023/040080
6. Campolo et al., A Review of the Containers Available for Multi-Dose Preservative-Free Eye Drops, Biomed J Sci & Tech Res: 45 (1)-2022, doi.org/10.26717/BJSTR.2022.45.007130
7. Aptar Product Brochure-published 3/2026
8. Allingham M J et al. Opththamol Sci. 2022; 1:100095
9. Gautam, et al. NeurobiolDis. 2023; 178:106022
10. DiMatteo M, et al. Next-generation ocular topical SBT-272 demonstrates optimized retina tissue exposure: building upon elamipretide for treating dry AMD. Investigative Ophthalmology & Visual Science. 2024; 65 (7): 707
11. Abbruscato A, et al. Topical bevemipretide (SBT-272) eye drops in development for dry AMD: Phase 1 trial design informed by 13-week GLP study results. Investigative Ophthalmology & Visual Science. 2025; 66 (8): 2532
12. McGrady N R, et al. Neuroprotective effects of topical bevemipretide (SBT-272) in a rodent model of glaucoma. Investigative Ophthalmology & Visual Science. 2025; 66 (8): 3425
13. Vardy K, et al. Ocular topical bevemipretide: targeting retinal tissue and preventing RPE cell and photoreceptor loss in a dry age-related macular degeneration (AMD) model. Investigative Ophthalmology & Visual Science. 2025; 66 (8)
14. WO 2019/118878 A1
15. WO 2021/016462 A1
16. WO 2023/069549 A1
17. WO 2023/224989 A1
18. WO 2024/092154 A1
19. WO 2024/243490 A1
20. WO 2024/254164 A1
21. WO 2026/050137 A1
22. US 2025/0302909 A1

What is claimed is:

1. A preservative-free ophthalmic topical composition comprising:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

2. An ophthalmic topical composition, the composition comprising an aqueous solution prepared by the dissolution in water of:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
sufficient sodium hydroxide and/or hydrochloric acid to maintain a desired pH of the composition;
wherein, the desired pH is in the range of 5.7 to 5.9, inclusive.

3. The ophthalmic topical composition of claim 1 or 2, wherein the composition comprises:
about 1% bevemipretide;
about 5.3 mg/mL NaCl; and
about 4.16 mg/mL NaH₂PO₄·H₂O;
wherein the pH is about 5.8.

4. The ophthalmic topical composition of claim 1 or 2, wherein the composition comprises:
about 2% bevemipretide;
about 3.4 mg/mL NaCl; and
about 4.16 mg/mL NaH₂PO₄·H₂O;
wherein the pH is about 5.8.

5. The ophthalmic topical composition of claim 1 or 2, wherein the composition comprises:
about 3% bevemipretide;
about 1.5 mg/mL NaCl; and
about 4.16 mg/mL NaH₂PO₄·H₂O;
wherein the pH is about 5.8.

6. The ophthalmic topical composition of any one of claims 1 to 5, wherein the composition is sterile.

7. An ophthalmic topical composition consisting essentially of:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

8. The ophthalmic topical composition of claim 7, wherein the composition consists essentially of:
about 1% bevemipretide;
about 5.3 mg/mL NaCl; and
about 4.16 mg/mL NaH₂PO₄·H₂O;
wherein the pH is about 5.8.

9. The ophthalmic topical composition of claim 7, wherein the composition consists essentially of:
about 2% bevemipretide;
about 3.4 mg/mL NaCl; and
about 4.16 mg/mL NaH₂PO₄·H₂O;
wherein the pH is about 5.8.

10. The ophthalmic topical composition of claim 7, wherein the composition consists essentially of:
about 3% bevemipretide;
about 1.5 mg/mL NaCl; and
about 4.16 mg/mL NaH₂PO₄·H₂O;
wherein the pH is about 5.8.

11. The ophthalmic topical composition of any one of claims 7 to 10, wherein the composition is sterile.

12. The ophthalmic topical composition of any one of claims 7 to 11, wherein the composition is preservative-free.

13. A packaged ophthalmic topical composition comprising a blow-fill-seal (BFS) vial, wherein the blow-fill-seal vial comprises a composition comprising:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

14. The packaged ophthalmic topical composition of claim 13, wherein the composition comprises:
about 1% bevemipretide;
about 5.3 mg/mL NaCl; and
about 4.16 mg/mL NaH₂PO₄·H₂O;
wherein the pH is about 5.8.

15. The packaged ophthalmic topical composition of claim 13, wherein the composition comprises:
about 2% bevemipretide;
about 3.4 mg/mL NaCl; and
about 4.16 mg/mL NaH₂PO₄·H₂O;
wherein the pH is about 5.8.

16. The packaged ophthalmic topical composition of claim 13, wherein the composition comprises:
about 3% bevemipretide;
about 1.5 mg/mL NaCl; and
about 4.16 mg/mL NaH₂PO₄·H₂O;
wherein the pH is about 5.8.

17. The packaged ophthalmic topical composition of any one of claims 13 to 16, wherein a volume of the blow-fill-seal vial is in the range of 0.5 mL to 1.0 mL, inclusive.

18. The packaged ophthalmic topical composition of claim 17, wherein the volume of the blow-fill-seal vial is about 0.8 mL.

19. The packaged ophthalmic topical composition of claim 17 or 18, wherein a volume of the composition in the blow-fill-seal vial is in the range of 0.24 mL to 0.40 mL, inclusive.

20. The packaged ophthalmic topical composition of claim 19, wherein the volume of the composition in the blow-fill-seal vial is about 0.32 mL.

21. The packaged ophthalmic topical composition of any one of claims 13 to 20, wherein the blow-fill-seal vial is linked physically to one more additional blow-fill-seal vials also containing the ophthalmic topical composition.

22. The packaged ophthalmic topical composition of claim 21, wherein the blow-fill-seal vial is linked physically to 4 additional blow-fill-seal vials also containing the ophthalmic topical composition.

23. The packaged ophthalmic topical composition of claim 21, wherein the blow-fill-seal vial is linked physically to 6 additional blow-fill-seal vials also containing the ophthalmic topical composition.

24. The packaged ophthalmic topical composition of claim 21, wherein the blow-fill-seal vial is linked physically to 13 additional blow-fill-seal vials also containing the ophthalmic topical composition.

25. The packaged ophthalmic topical composition of claim 21, wherein the blow-fill-seal vial is linked physically to 27 additional blow-fill-seal vials also containing the ophthalmic topical composition.

26. The packaged ophthalmic topical composition of claim 21, wherein the blow-fill-seal vial is linked physically to 29 or 30 additional blow-fill-seal vials also containing the ophthalmic topical composition.

27. The packaged ophthalmic topical composition of claim 21, wherein the blow-fill-seal vial is linked physically to 1 to 30 additional blow-fill-seal vials also containing the ophthalmic topical composition.

28. The packaged ophthalmic topical composition of any one of claims 13 to 27, wherein the blow-fill-seal vial comprises low density polyethylene (LDPE).

29. The packaged ophthalmic topical composition of any one of claims 13 to 28, wherein the blow-fill-seal-vial comprises a twist off cap.

30. The packaged ophthalmic topical composition of any one of claims 13 to 29, wherein the composition is sterile.

31. The packaged ophthalmic topical composition of any one of claims 13 to 30, wherein the composition is preservative-free.

32. The packaged ophthalmic topical composition of any one of claims 13 to 31, wherein the blow-fill-seal-vial is adapted for single use.

33. A packaged ophthalmic topical composition comprising a multi-dose container,
wherein the multi-dose container comprises a composition comprising:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and water;
wherein a pH of the ophthalmic topical composition is in the range of 5.7 to 5.9, inclusive.

34. The packaged ophthalmic topical composition of claim 33, wherein the composition comprises:
about 1% bevemipretide;
about 5.3 mg/mL NaCl; and
about 4.16 mg/mL NaH₂PO₄·H₂O;
wherein the pH is about 5.8.

35. The packaged ophthalmic topical composition of claim 33, wherein the composition comprises:
about 2% bevemipretide;
about 3.4 mg/mL NaCl; and
about 4.16 mg/mL NaH₂PO₄·H₂O;
wherein the pH is about 5.8.

36. The packaged ophthalmic topical composition of claim 33, wherein the composition comprises:
about 3% bevemipretide;
about 1.5 mg/mL NaCl; and
about 4.16 mg/mL NaH₂PO₄·H₂O;
wherein the pH is about 5.8.

37. The packaged ophthalmic topical composition of any one of claims 33 to 36, wherein the composition is preservative-free.

38. The packaged ophthalmic topical composition of any one of claims 33 to 37, wherein the composition is sterile.

39. The packaged ophthalmic topical composition of claim 38, wherein the multi-dose container is adapted to maintain sterility of the composition for a week or more, a month or more, three months or more, 6 months or more or a year or more.

40. The packaged ophthalmic topical composition of any one of claims 33 to 39, wherein the multi-dose container is adapted to comprise between 2.5 mL and 30 mL, inclusive, of ophthalmic topical composition.

41. The packaged ophthalmic topical composition of any one of claims 33 to 40, wherein the multi-dose container is adapted to deliver a consistent dose of between 25 μL and 35 μL, inclusive, as a drop of ophthalmic topical solution each time it is used.

42. The packaged ophthalmic topical composition of claim 41, wherein the multi-dose container is adapted to deliver a consistent dose of about 30 μL as a drop of ophthalmic topical solution each time it is used.

43. A single-use blow-fill-seal (BFS) vial comprising an ophthalmic topical composition comprising:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

44. A single-use blow-fill-seal (BFS) vial comprising an ophthalmic topical composition consisting essential of:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

45. The single-use blow-fill-seal (BFS) vial of claim 43 or 44 comprises or consists essentially of:
about 1% bevemipretide;
about 5.3 mg/mL NaCl; and
about 4.16 mg/mL NaH₂PO₄·H₂O;
wherein the pH is about 5.8.

46. The single-use blow-fill-seal (BFS) vial of claim 43 or 44 comprises or consists essentially of:
about 2% bevemipretide;
about 3.4 mg/mL NaCl; and
about 4.16 mg/mL NaH₂PO₄·H₂O;
wherein the pH is about 5.8.

47. The single-use blow-fill-seal (BFS) vial of claim 43 or 44 comprises or consists essentially of:
about 3% bevemipretide;
about 1.5 mg/mL NaCl; and
about 4.16 mg/mL NaH₂PO₄·H₂O;
wherein the pH is about 5.8.

48. The single-use blow-fill-seal (BFS) vial of any one of claims 43 to 47, wherein a volume of the blow-fill-seal vial is in the range of 0.5 mL to 1.0 mL, inclusive.

49. The single-use blow-fill-seal (BFS) vial of claim 48, wherein the volume of the blow-fill-seal vial is about 0.8 mL.

50. The single-use blow-fill-seal (BFS) vial of any one of claims 43 to 49, wherein a volume of the composition in the blow-fill-seal vial is in the range of 0.24 mL to 0.40 mL, inclusive.

51. The single-use blow-fill-seal (BFS) vial of claim 50, wherein the volume of the composition in the blow-fill-seal vial is about 0.32 mL.

52. The single-use blow-fill-seal (BFS) vial of any one of claims 43 to 51, wherein the composition is sterile.

53. The single-use blow-fill-seal (BFS) vial of any one of claims 43 to 52, wherein the composition is preservative-free.

54. A multi-dose container comprising an ophthalmic topical composition comprising:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

55. A multi-dose container comprising an ophthalmic topical composition consisting essential of:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and
water;
wherein a pH of the ophthalmic topical composition is in the range of 5.7 to 5.9, inclusive.

56. The multi-dose container of claim 54 or 55 comprising or consisting essentially of:
about 1% bevemipretide;
about 5.3 mg/mL NaCl; and
about 4.16 mg/mL NaH₂PO₄·H₂O;
wherein the pH is about 5.8.

57. The multi-dose container of claim 54 or 55 comprising or consisting essentially of:
about 2% bevemipretide;
about 3.4 mg/mL NaCl; and
about 4.16 mg/mL NaH₂PO₄·H₂O;
wherein the pH is about 5.8.

58. The multi-dose container of claim 54 or 55 comprising or consisting essentially of:
about 3% bevemipretide;
about 1.5 mg/mL NaCl; and
about 4.16 mg/mL NaH₂PO₄·H₂O;
wherein the pH is about 5.8.

59. The multi-dose container of any one of claims 54 to 58, wherein the composition is preservative-free.

60. The multi-dose container of any one of claims 54 to 59, wherein the composition is sterile.

61. The multi-dose container of claim 60, wherein the multi-dose container is adapted to maintain sterility of the composition for a week or more, a month or more, three months or more, 6 months or more or a year or more.

62. The multi-dose container of any one of claims 54 to 61, wherein the multi-dose container is adapted to comprises between 2.5 mL and 30 mL, inclusive, of ophthalmic topical composition.

63. The multi-dose container of any one of claims 54 to 62, wherein the multi-dose container is adapted to deliver a consistent dose of between 25 μL and 35 μL, inclusive, as a drop of ophthalmic topical solution each time it is used.

64. The multi-dose container of claim 63, wherein the multi-dose container is adapted to deliver a consistent dose of about 30 μL as a drop of ophthalmic topical solution each time it is used.

65. A method of preparing a preservative-free composition comprising bevemipretide or a pharmaceutically acceptable salt thereof, the method comprising:
dissolving in an aqueous medium:
about 1% to about 3% bevemipretide, or a pharmaceutically acceptable salt thereof, where the percent bevemipretide is based on the free-base weight of the bevemipretide to volume (w/v) of the composition,
about 1.4 mg/mL to about 5.4 mg/mL NaCl; and
about 4.1 mg/mL to about 4.2 mg/mL NaH₂PO₄·H₂O; and
adding sodium hydroxide or hydrochloric acid in amount sufficient to adjust pH of the ophthalmic topical composition to the range of 5.7 to 5.9, inclusive.

66. The method of claim 65, wherein the composition comprises:
about 1% bevemipretide;
about 5.3 mg/mL NaCl; and
about 4.16 mg/mL NaH₂PO₄·H₂O; and wherein the pH is about 5.8.

67. The method of claim 65, wherein the composition comprises:
about 2% bevemipretide;
about 3.4 mg/mL NaCl; and
about 4.16 mg/mL NaH₂PO₄·H₂O; wherein the pH is about 5.8.

68. The method of claim 65, wherein the composition comprises:
about 3% bevemipretide;
about 1.5 mg/mL NaCl; and
about 4.16 mg/mL NaH₂PO₄·H₂O; wherein the pH is about 5.8.

69. The method of any one of claims 65 to 68, wherein the composition is sterile.

70. The method of claim 69, wherein after preparation, a portion of the sterile composition is transferred to a blow-fill-seal (BFS) vial.

71. The method of claim 70, wherein the blow-fill-seal vial is adapted for single-use.

72. The method of claim 71, wherein the volume of the single-use blow-fill-seal vial is in the range of 0.5 mL to 1.0 mL, inclusive.

73. The method of claim 72, wherein the volume of the single-use blow-fill-seal vial is about 0.8 mL.

74. The method of claim 72 or 73, wherein the volume of the composition in the single-use blow-fill-seal vial is in the range of 0.24 mL to 0.40 mL, inclusive.

75. The method of claim 74, wherein the volume of the composition in the single-use blow-fill-seal vial is about 0.32 mL.

76. The method of claim 69, wherein after preparation, a portion of the sterile composition is transferred to a multi-dose container.

77. The method of claim 76, wherein the multi-dose container is adapted to maintain sterility of the composition for a week or more, a month or more, three months or more, 6 months or more or a year or more.

78. The method of claim 76 or 77, wherein the multi-dose container is adapted to comprises between 2.5 mL and 30 mL, inclusive, of the composition.

79. The method of any one of claims 76 to 78, wherein the multi-dose container is adapted to deliver a consistent dose of between 25 μL and 35 μL, inclusive, as a drop of the composition each time it is used.

80. The method of claim 79, wherein the multi-dose container is adapted to deliver a consistent dose of about 30 μL as a drop of the composition each time it is used.

81. A method of dosing bevemipretide, or a pharmaceutically acceptable salt thereof, to a subject in need thereof, the method comprising:
administering a preservative-free ophthalmic topical composition once daily, twice daily, or three times daily to the subject;
wherein the ophthalmic topical composition comprises:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl; and
about 4.1 mg/mL to about 4.2 mg/mL NaH₂PO₄·H₂O; and water;
wherein pH of the ophthalmic topical composition is in a range of 5.7 to 5.9, inclusive.

82. The method of claim 81, wherein the subject has been diagnosed with macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), or oxygen-induced retinopathy.

83. The method of claim 81, wherein the subject has been diagnosed with macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy.

84. The method of claim 81, wherein the subject has been diagnosed with dry age-related macular degeneration, glaucoma, pigmentary retinopathy, retinitis pigmentosa, diabetic retinopathy, diabetic macular edema, Stargardt's disease, or oxygen-induced retinopathy.

85. A method of dosing bevemipretide, or a pharmaceutically acceptable salt thereof, to a subject in need thereof, the method comprising:
administering an ophthalmic topical composition once daily, twice daily, or three times daily to the subject;
wherein the ophthalmic topical composition consists essentially of:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and water;
wherein pH of the ophthalmic topical composition is in a range of 5.7 to 5.9, inclusive.

86. The method of claim 85, wherein the subject has been diagnosed with macular degeneration, dry age-related macular degeneration, wet age-related macular degeneration, diabetic retinopathy, diabetic macular edema, dry-eye, cataracts, autosomal dominant optic atrophy (DOA), Leber hereditary optic neuropathy (LHON), pigmentary retinopathy, retinitis pigmentosa, glaucoma, ocular hypertension, choroideremia, uveitis, choroidal neovascularization, chronic progressive external ophthalmoplegia (e.g., Kearns-Sayre syndrome), Stargardt's disease, Leber congenital amaurosis (LCA), or oxygen-induced retinopathy.

87. The method of claim 85, wherein the subject has been diagnosed with macular degeneration, dry age-related macular degeneration, wet age-related macular degeneration, diabetic retinopathy, diabetic macular edema, autosomal dominant optic atrophy (DOA), Leber hereditary optic neuropathy (LHON), pigmentary retinopathy, retinitis pigmentosa, glaucoma, ocular hypertension, choroideremia, uveitis, Stargardt's disease, Leber congenital amaurosis (LCA), or oxygen-induced retinopathy.

88. The method of claim 85, wherein the subject has been diagnosed with dry age-related macular degeneration, glaucoma, pigmentary retinopathy, retinitis pigmentosa, diabetic retinopathy, diabetic macular edema, Stargardt's disease, or oxygen-induced retinopathy.

89. A method for treating, preventing, inhibiting, ameliorating, or delaying the onset of dry age-related macular degeneration in a subject in need thereof, the method comprising:
administering a preservative-free composition to the subject at least once daily, wherein the composition comprises:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl;
about 4.1 mg/mL to about 4.2 mg/mL NaH₂PO₄·H₂O; and water;
wherein pH of the ophthalmic topical composition is in the range of 5.7 to 5.9, inclusive.

90. The method of claim 89, wherein the administering is conducted once daily, twice daily, or three times daily.

91. A method for treating, preventing, inhibiting, ameliorating, or delaying the onset of dry age-related macular degeneration in a subject in need thereof, the method comprising:
administering a composition to the subject at least once daily, the composition consisting essentially of:
about 1% to about 3% bevemipretide or a pharmaceutically acceptable salt thereof, where the percent bevemipretide is based on the free-base weight of the bevemipretide to volume (w/v) of the composition;
about 1.4 mg/mL to about 5.4 mg/mL NaCl; and
about 4.1 mg/mL to about 4.2 mg/mL NaH₂PO₄·H₂O; and water;
wherein pH of the ophthalmic topical composition is in the range of 5.7 to 5.9, inclusive.

92. The method of claim 91, wherein the administering is conducted once daily, twice daily, or three times daily.

93. The method of any one of claims 81 to 92, wherein the ophthalmic topical composition is administered using a blow-fill-seal (BFS) vial.

94. The method of claim 93, wherein the blow-fill-seal (BFS) vial is adapted for single-use.

95. The method of any one of claims 81 to 92, wherein the ophthalmic topical composition is administered using a multi-dose container.

96. The method of claim 95, wherein multi-dose container comprises between 2.5 mL and 30 mL, inclusive, of ophthalmic topical composition.

97. The method of any one of claims 81 to 96, wherein the ophthalmic topical composition is administered to one eye of the subject.

98. The method of any one of claims 81 to 96, wherein the ophthalmic topical composition is administered to both eyes of the subject.

99. The method of any one of claims 81 to 98, wherein the ophthalmic topical composition is administered at a rate of one drop per eye of the subject per administration.

100. The method of any one of claims 81 to 98, wherein the ophthalmic topical composition is administered at a rate of two or more drops per eye of the subject per administration.

101. The method of any one of claims 81 to 100, wherein each drop administered to the eye is in the range of 22 μL to 38 μL, inclusive, in volume.

102. The method of claim 101, wherein each drop is about 30 μL in volume.

103. The method of any one of claims 81 to 102, wherein the administering is repeated daily for at least 12 weeks, at least 24 weeks, at least 36 weeks, at least 48 weeks, at least 60 weeks, at least 72 weeks, at least 84 weeks, or at least 96 weeks.

104. The method of any one of claims 81 to 103, wherein the composition comprises or consists essentially of:
about 1% bevemipretide;
about 5.3 mg/mL NaCl; and
about 4.16 mg/mL NaH₂PO₄·H₂O; wherein the pH is about 5.8.

105. The method of any one of claims 81 to 103, wherein the composition comprises or consists essentially of:
about 2% bevemipretide;
about 3.4 mg/mL NaCl; and
about 4.16 mg/mL NaH₂PO₄·H₂O; wherein the pH is about 5.8.

106. The method of any one of claims 81 to 103, wherein the composition comprises or consists essentially of:
about 3% bevemipretide;
about 1.5 mg/mL NaCl; and
about 4.16 mg/mL NaH₂PO₄·H₂O; wherein the pH is about 5.8.

107. The method of any one of claims 81 to 106, wherein the subject has been diagnosed with or is at risk for developing dry age-related macular degeneration.

108. The method of claim 107, wherein the subject has drusen.

109. The method of claim 107 or 108, wherein the subject has been diagnosed with or is at risk for developing geographic atrophy (GA).

110. The method of any one of claims 81 to 109, wherein the subject has been diagnosed with partial ellipsoid zone attenuation of one or more portions/zones of the macula of at least one eye.

111. The method of any one of claims 81 to 110, wherein the subject has been diagnosed with total ellipsoid zone attenuation of one or more portions/zone of the macula of at least one eye.

112. The method of any one of claims 81 to 111, wherein the administration of the ophthalmic topical composition is administered to the subject once per day.

113. The method of any one of claims 81 to 111, wherein the administration of the ophthalmic topical composition is administered to the subject twice per day

114. The method of any one of claims 81 to 111, wherein the administration of the ophthalmic topical composition is administered to the subject three times per day.

115. The method of any one of claims 81 to 114, wherein the ophthalmic topical composition is sterile.

116. The method of any one of claims 81 to 115, wherein the subject is human.

117. A preservative-free ophthalmic topical composition comprising:
about 1% bevemipretide, or a pharmaceutically acceptable salt thereof, where the percent bevemipretide is based on the free-base weight of the bevemipretide to volume (w/v) of the composition;
about 5.3 mg/mL NaCl; and
about 4.16 mg/mL NaH₂PO₄·H₂O; wherein the pH is about 5.8.

118. A preservative-free ophthalmic topical composition comprising:
about 2% bevemipretide, or a pharmaceutically acceptable salt thereof, where the percent bevemipretide is based on the free-base weight of the bevemipretide to volume (w/v) of the composition;
about 3.4 mg/mL NaCl; and
about 4.16 mg/mL NaH₂PO₄·H₂O; wherein the pH is about 5.8.

119. A preservative-free ophthalmic topical composition comprising:
about 3% bevemipretide, or a pharmaceutically acceptable salt thereof, where the percent bevemipretide is based on the free-base weight of the bevemipretide to volume (w/v) of the composition;
about 1.5 mg/mL NaCl; and
about 4.16 mg/mL NaH₂PO₄·H₂O; wherein the pH is about 5.8.

120. An ophthalmic topical composition consisting essentially of:
about 1% bevemipretide, or a pharmaceutically acceptable salt thereof, where the percent bevemipretide is based on the free-base weight of the bevemipretide to volume (w/v) of the composition;
about 5.3 mg/mL NaCl; and
about 4.16 mg/mL NaH₂PO₄·H₂O; wherein the pH is about 5.8.

121. An ophthalmic topical composition consisting essentially of:
about 2% bevemipretide, or a pharmaceutically acceptable salt thereof, where the percent bevemipretide is based on the free-base weight of the bevemipretide to volume (w/v) of the composition;
about 3.4 mg/mL NaCl; and
about 4.16 mg/mL NaH₂PO₄·H₂O; wherein the pH is about 5.8.

122. An ophthalmic topical composition consisting essentially of:
about 3% bevemipretide, or a pharmaceutically acceptable salt thereof, where the percent bevemipretide is based on the free-base weight of the bevemipretide to volume (w/v) of the composition;
about 1.5 mg/mL NaCl; and
about 4.16 mg/mL NaH₂PO₄·H₂O; wherein the pH is about 5.8.

123. The ophthalmic topical composition of any one of claims 117 to 122, wherein the composition is sterile.

124. The ophthalmic topical composition of claim 123 packaged in a single-use blow-fill-seal vial.

125. The ophthalmic topical composition of claim 123 packaged in a multi-dose container adapted to maintain sterility of the composition for a week or more, a month or more, three months or more, 6 months or more or a year or more.

126. The ophthalmic topical composition of any one of claims 117 to 125, wherein the osmolality of the composition is in the range of 260-285 mOsm/kg, inclusive.

127. A method of dosing bevemipretide, or a pharmaceutically acceptable salt thereof, to a subject in need thereof, the method comprising:
administering as an eye drop an ophthalmic topical composition once daily or twice daily to the subject;
wherein the ophthalmic topical composition comprises:
about 1% bevemipretide, or a pharmaceutically acceptable salt thereof, where the percent bevemipretide is based on the free-base weight of the bevemipretide to volume (w/v) of the composition;
about 5.3 mg/mL NaCl; and
about 4.16 mg/mL NaH₂PO₄·H₂O; wherein the pH is about 5.8.

128. A method of dosing bevemipretide, or a pharmaceutically acceptable salt thereof, to a subject in need thereof, the method comprising:
administering as an eye drop an ophthalmic topical composition once daily or twice daily to the subject;
wherein the ophthalmic topical composition comprises:
about 2% bevemipretide, or a pharmaceutically acceptable salt thereof, where the percent bevemipretide is based on the free-base weight of the bevemipretide to volume (w/v) of the composition;
about 3.4 mg/mL NaCl; and
about 4.16 mg/mL NaH₂PO₄·H₂O; wherein the pH is about 5.8.

129. A method of dosing bevemipretide, or a pharmaceutically acceptable salt thereof, to a subject in need thereof, the method comprising:
administering as an eye drop an ophthalmic topical composition once daily or twice daily to the subject;
wherein the ophthalmic topical composition comprises:
about 3% bevemipretide, or a pharmaceutically acceptable salt thereof, where the percent bevemipretide is based on the free-base weight of the bevemipretide to volume (w/v) of the composition;
about 1.5 mg/mL NaCl; and
about 4.16 mg/mL NaH₂PO₄·H₂O; wherein the pH is about 5.8.

130. The method of any one of claims 127 to 129, wherein the ophthalmic topical composition is preservative-free.

131. The method of any one of claims 127 to 130, wherein the ophthalmic topical composition is sterile.

132. The method of any one of claims 127 to 131, wherein the subject is human.

133. The method of any one of claims 127 to 132, wherein the ophthalmic topical composition is packaged in a single-use blow-fill-seal vial.

134. The method of any one of claims 127 to 132, wherein the ophthalmic topical composition is packaged in a multi-dose container adapted to maintain sterility of the composition for a week or more, a month or more, three months or more, 6 months or more or a year or more.

135. The method of any one of claims 127 to 134, wherein the dosing is repeated daily for at least 12 weeks, at least 24 weeks, at least 36 weeks, at least 48 weeks, at least 60 weeks, at least 72 weeks, at least 84 weeks, or at least 96 weeks.

136. A method of dosing (R)-2-amino-N-((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide (bevemipretide), or a pharmaceutically acceptable salt, stereoisomer, tautomer, hydrate, and/or solvate thereof, in a subject in need thereof, the method comprising:
(i) administering an ophthalmic topical composition comprising 1% (wt./vol.) bevemipretide, or a pharmaceutically acceptable salt, stereoisomer, tautomer, hydrate, and/or solvate thereof, once daily, twice daily, or three times daily to the subject;
(ii) administering an ophthalmic topical composition comprising 2% (wt./vol.) bevemipretide, or a pharmaceutically acceptable salt, stereoisomer, tautomer, hydrate, and/or solvate thereof, once daily, twice daily, or three times daily to the subject; or
(iii) administering an ophthalmic topical composition comprising 3% (wt./vol.) bevemipretide, or a pharmaceutically acceptable salt, stereoisomer, tautomer, hydrate, and/or solvate thereof, once daily, twice daily, or three times daily to the subject.

137. A method for treating, preventing, inhibiting, ameliorating, or delaying the onset of dry age-related macular degeneration, the method comprising dosing (R)-2-amino-N-((S)-1-(((S)-5-amino-1-(3-benzyl-1,2,4-oxadiazol-5-yl) pentyl)amino)-3-(4-hydroxy-2,6-dimethylphenyl)-1-oxopropan-2-yl)-5-guanidinopentanamide (bevemipretide), or a pharmaceutically acceptable salt, stereoisomer, tautomer, hydrate, and/or solvate thereof, in a subject in need thereof, the method comprising:
(i) administering an ophthalmic topical composition comprising 1% (wt./vol.) bevemipretide, or a pharmaceutically acceptable salt, stereoisomer, tautomer, hydrate, and/or solvate thereof, once daily, twice daily, or three times daily to the subject;
(ii) administering an ophthalmic topical composition comprising 2% (wt./vol.) bevemipretide, or a pharmaceutically acceptable salt, stereoisomer, tautomer, hydrate, and/or solvate thereof, once daily, twice daily, or three times daily to the subject; or
(iii) administering an ophthalmic topical composition comprising 3% (wt./vol.) bevemipretide, or a pharmaceutically acceptable salt, stereoisomer, tautomer, hydrate, and/or solvate thereof, once daily, twice daily, or three times daily to the subject.

138. The method of claim 136 or claim 137, wherein the subject has been diagnosed with or is at risk for developing dry age-related macular degeneration.

139. The method of any one of claims 136-138, wherein the subject has drusen.

140. The method of any one of claims 136-139, wherein the subject has been diagnosed with or is at risk for developing geographic atrophy (GA).

141. The method of any one of claims 136-140, wherein the ophthalmic topical composition is an eye drop.

142. The method of any one of claims 136-141, wherein the ophthalmic topical composition is a buffered sterile solution.

143. The method of any one of claims 136-142, wherein the subject is human.

144. The method of any one of claims 136-143, wherein the dosing is repeated daily for at least 12 weeks, at least 24 weeks, at least 36 weeks, at least 48 weeks, at least 60 weeks, at least 72 weeks, at least 84 weeks or at least 96 weeks.
